(19) United States
(12) Patent Application Publication
(10) Pub. No.: US 2021/0033887 A1
(43) Pub. Date: Feb. 4, 2021

(54) SYSTEMS AND METHODS FOR FORMING OPHTHALMIC LENS INCLUDING META OPTICS

(71) Applicant: MENICON CO., LTD., Nagoya (JP)

(72) Inventor: Stephen D. Newman, Singapore (SG)

(21) Appl. No.: 16/942,403

(22) Filed: Jul. 29, 2020

Related U.S. Application Data
(60) Provisional application No. 62/879,834, filed on Jul. 29, 2019.

(51) Int. Cl. G02C 7/04; G02B 1/18; B29D 11/00; A61F 2/16

(52) U.S. Cl. CPC G02C 7/049; G02B 1/18; B29D 11/0048; B29K 2105/0061; A61F 2/1613; G02C 2202/22; G02C 2202/24; B29D 11/00038

(57) ABSTRACT

Ophthalmic lenses and methods of manufacture thereof are disclosed. The ophthalmic lenses include metasurface features that define a metasurface array on a lens body. The metasurface array can be tuned to modify an optical property, such as glare reduction, of the ophthalmic lens using an arrangement of metasurface building elements dimensioned from an optical wavelength, including being dimensioned smaller than an optical wavelength. The ophthalmic lenses can be subject to physical manipulation, including rolling or other folding during an installation procedure, and as such, the modified optical property induced by the metasurface array can be maintained after such manipulation. The ophthalmic lenses can be formed using a molding process, in which the metasurface array is associated with a non-solid material and formed into a lens shape with the material.

### CROSS-REFERENCE TO RELATED APPLICATIONS

This patent application is a non-provisional patent application of, and claims priority to, U.S. Provisional Application No. 62/879,834 filed Jul. 29, 2019, and titled “SYSTEMS AND METHODS FOR FORMING OPHTHALMIC LENS INCLUDING META OPTICS FIELD,”; the disclosure of which is hereby incorporated by reference in its entirety.

### FIELD

The described embodiments relate generally to ophthalmic devices, and more particularly, to systems and techniques for modifying optical properties of a lens using metasurface features.

### BACKGROUND

Ophthalmic devices can be used to provide vision correction to a user, treat various diseases, and so on. In many traditional applications, the geometry of the device itself is used to induce a desired optical characteristic of a lens body associated with the treatment, such as via refraction. Many traditional systems suffer from significant drawbacks as the physical properties and dimensions of the device can be limited by the desired optical characteristic. This can create unduly bulky ophthalmic devices that can decrease user compliance and adaptability to certain surgical techniques and/or other use cases. As such, the need continues for systems and techniques to facilitate ophthalmic devices being geometrically unconstrained by a desired optical characteristic.

### SUMMARY

Embodiments of the present invention are directed to ophthalmic devices or lenses and methods of manufacturing thereof. The ophthalmic lenses can have a metasurface array that defines one or more metasurface features with a lens body. The metasurface features can operate to modify an optical property of the lens, including modifying a focal point, an aberration characteristic, a glare/halo characteristic, and/or other properties, which can be associated with vision correction. The metasurface array can also operate to define focal distances relative to respective meridians of the lens, such as having a first focal distance associated with a first meridian and a second focal distance associated with a second meridian, as contemplated herein. The metasurface features can be used to modify the optical property of the lens without relying on techniques dependent on the geometry of the lens body itself to produce an optical effect. In this manner, the ophthalmic lenses of the present disclosure can have a desired optical effect without necessarily relying on the geometry of the lens body, thus enhancing design versatility and expanding manufacturing possibilities, including the standardization of lens substrate designs.

To facilitate the foregoing, the metasurface features can be defined by a metasurface array associated with a lens body. Broadly, the metasurface array can be configured to shift a phase of incident light, this can be accomplished using resonance-based effects, including electrical and magnetic-type resonance effects. In other cases, the metasurface array can employ the Pancharatnam-Berry phase to facilitate light modification. In other cases, other techniques can be used to shift a phase of incident light. To facilitate the foregoing, the metasurface array can have an arrangement of metasurface building elements. The arrangement of metasurface building elements can be specifically tuned to interact with light traversing the associated lens body to induce a desired optical effect in the ophthalmic device. For example, the metasurface building elements can be dimensioned of, or smaller than, an optical wavelength, such as a cycle wavelength of light. The metasurface building elements can also be physically arranged in a variety of configurations, including having metasurface building elements of different sizes, groupings, orientations, densities, and so forth. As such, optical wavelengths traversing the associated lens body exhibit characteristics influenced by the metasurface and the specific arrangement of the elements on the lens body. This arrangement can be tuned to induce a desired optical characteristic, as outlined herein, including inducing a desired vision correction.

While many examples are disclosed herein, in one embodiment, an ophthalmic lens is disclosed. The ophthalmic lens includes a lens body. The ophthalmic lens further includes a metasurface array on the lens body having an arrangement of metasurface building elements dimensioned from an optical wavelength and configured across the lens body to define a reduced glare characteristic of the ophthalmic lens. The reduced glare characteristic is maintained after physically manipulating the ophthalmic lens for use with the eye.

Additionally or alternatively, other optical properties of the ophthalmic lens can be modified using the arrangement of metasurface building elements described herein. For example, in one embodiment, the arrangement of metasurface building elements are configured across the lens body for halo reduction of the ophthalmic lens. Further, the arrangement of metasurface building elements can be configured across the lens body for contrast enhancement of the ophthalmic lens. The contrast enhancement can be measured based on a variety of tests, including the Cambridge low-contrast grating test, the CSV-1000 test, the Pelli-Robson test, and/or the Mars letters test, among others. Although specific values of the contrast may vary based on population, the ophthalmic devices of the present disclosure may enhance the contrast value, using one or more these scales, by as much as 5%, by as much as 10%, by as much as 15%, or greater.

Aberration characteristics can also be modified and corrected. For example, in another embodiment, the arrangement of metasurface building elements are configured across the lens body to reduce an aberration characteristic of the lens body. The aberration characteristic can include one or both of a chromatic aberration or a monochromatic aberration. Visual enhancement is also contemplated herein using the metasurface building elements.

In some cases, the lens body can be a wide-angle contact lens body. The metasurface building elements can include dimensions less than a wavelength of light traversing the lens body. The dimensions of the metasurface building elements can include a height dimension of the metasurface building elements.

In another embodiment, the metasurface building elements can include a collection of nano-posts. The collection of nano-posts can include nano-posts of dissimilar shapes. Further, the collection of nano-posts can include nano-posts of dissimilar orientations. In some cases, the collection of nano-posts can define a first density of metasurface building elements on a first portion of the lens body, and a second density of metasurface building elements on a second portion of the lens body that is different than the first density. The first and second densities can be arranged to induce the modified optical property.

In another embodiment, the optical property can include one or more focal points of the lens body. In this regard, the metasurface array can operate to induce optical properties associated with the bifocal, progressive multifocal and trifocal for vision correction.

In another embodiments, the optical property can include an astigmatism correcting property. In this regard, the metasurface array can operate to define or modify focal distances relative to respective meridians of the lens, such as having a first focal distance associated with a first meridian and a second focal distance associated with a second meridian.

In another embodiments, modifying the focal point can include modifying a decentralized focal point. In this regard, the metasurface array can operate to define the focal point as being decentralized relative a central axis of the lens. Additionally or alternatively, this can involve defining or modifying one or more focal points that focused at peripheral location disposed at a distance from fovea. In some cases, the modified focal point is configured to control myopia progression.

In another embodiments, metasurface features can combine with refraction and/or diffraction based optical zone. For example, the lens can include a central optic zone having metasurface structures, a peripheral optic zone surrounding central optic zone comprised by refraction and/or diffraction based optical property zone.

In another embodiment, the lens body can be associable with the eye. In this regard, the arrangement of metasurface building elements can be configured to provide vision correction for the eye. The physical manipulation can include rolling the ophthalmic lens for insertion into an incision of between about 1 mm and 2 mm. As explained herein, in other cases the incision can be less than 1 mm or greater than 2 mm, and the ophthalmic lens can be configured for insertion therethrough accordingly. The arrangement of metasurface building elements can be maintained after physically manipulating the lens body for use with an eye. The arrangement of metasurface building elements can also be maintained after folding the lens body for introduction to a region of the eye during surgery.

In another embodiment, the ophthalmic lens can be an intraocular lens (IOL). In some cases, the lens body can be substantially flat. The lens body can a portion with a thickness of about 0.25 mm; in some cases, the thickness can be more or less than 0.25 mm, as required for a given application. The thickness of the lens body and lens more generally can vary along one or more dimension of the lens. In this regard, to the extent that the lens body has a portion with a thickness of about 0.25 mm, this is not necessarily a uniform thickness. For example, an optical zone can have a thickness different from a thickness of the peripheral zone of the lens.

In another embodiment, the ophthalmic lens can be a contact lens. The contact lens can include one of a rigid gas permeable ocular lens or a scleral lens. In some cases, the contact lens can be a hybrid lens, including embodiments with a substantially soft periphery. Additionally or alternatively, the lens can include a hydrogel component, as may be appropriate for certain applications. In some cases, the contact lens can be a molded lens. Moreover, any of the ophthalmic lenses described herein can at least partially be formed from a titanium dioxide material. It will be appreciated that in other cases, other materials can be used and are contemplated herein.

In another embodiment, a method of manufacturing a foldable ophthalmic lens is disclosed. The method includes forming a metasurface array by establishing metasurface building elements in a matrix. The method further includes forming a lens body having a profile shaped to match a geometry of an eye. The method further includes associating the metasurface array with the lens body to form the foldable ophthalmic lens. The foldable ophthalmic lens is foldable or rollable for introduction through an incision and into a region of the eye during surgery. The metasurface is adapted to establish at least one of a low aberration characteristic, a low glare characteristic, or an enhanced contrast characteristic of the foldable ophthalmic lens in an installed configuration with the eye.

The foldable ophthalmic lens having the formed metasurface is adapted for insertion into and through a substantially small region of the eye for surgical association with the eye. In some cases, the foldable lens is adapted for insertion into and through an incision having of a size of 2.0 mm or less, 1.5 mm or less, or a smaller incision.

The foldable ophthalmic lens is insertable through the incision and configured to modify an optical characteristic of the eye notwithstanding the folding, rolling or other physical manipulation of the lens as the lens is advanced through the incisions. The lens body can also be adapted to facilitate the physical manipulation of the lens, including having such characteristics as being substantially flat prior to being folded or rolled for the introduction through the incision. In this regard and in some cases, the foldable ophthalmic lens can be an intraocular device having a diameter of less than about 6 mm and a thickness of less than about 0.25 mm, which may facilitate the introduction through the incision.

In another embodiment, the operation of associating includes coupling the metasurface array with a non-solid substrate. The non-solid substrate can include a precursor form of the lens body.

In another embodiment, the operation of associating the metasurface array and the lens body can be performed using a molding apparatus. The molding apparatus can include a first mold portion configured to receive the metasurface array. The molding apparatus can further include a second mold portion configured to press a lens material against the metasurface array. The lens material can include a liquid lens material defining a precursor form of the lens body. In this regard, the operation of forming the lens body can further include distributing the liquid lens material along the metasurface array by combining the first and second mold portions. In some cases, the operation of forming the lens body can further include curing the liquid lens material to form the lens body.

In another embodiment, the matrix can include a sacrificial matrix configured to be at least partially removed subsequent to the operation of associating. The operation of forming the metasurface array can include patterning an amorphous silicon layer to form nano-posts defining the metasurface building elements. The nano-posts can have a dimension of, or less than, an optical wavelength. In some cases, the operation of patterning comprises defining an arrangement of nano-posts tuned to the profile of the lens body. The operation of forming the metasurface array can further include one or both of lithography or dry etching. The operation of forming the metasurface array can further include associating the nano-posts with a matrix material forming the matrix. The matrix material can include polydimethylsiloxane, among other possible materials.

In another embodiment, the operation of forming the metasurface array includes forming a peelable sheet configured to adhere to an outer surface of the lens body during the operation of associating the metasurface array with the lens body.

In another embodiment, a method of manufacturing standardized ophthalmic lenses is disclosed. The method includes providing a group of standardized lens bodies. The method further includes producing a first ophthalmic lens by associating a first metasurface array with a first lens body of the group of standardized lens bodies. The method further includes producing a second ophthalmic lens by associating a second metasurface array with a second lens body of the group of standardized lens bodies. The first and second metasurface arrays have different arrangements of metasurface building elements, thereby inducing differential optical properties for the standardized bodies of the first and second ophthalmic lenses.

In another embodiment, the standardized lens bodies can have a portion with a thickness of about 0.25 mm or less. In some cases, the standardized lens bodies can be substantially flat.

In another embodiment, the standardized lens bodies can include a haptic feature for an intraocular lens. In other cases, the first and second ophthalmic devices are contact lenses, comprising a rigid gas permeable ocular lens or a scleral lens.

In another embodiment, the first ophthalmic lens can have a first focal point and the second ophthalmic lens can have a second focal point that is different from the first focal point. The metasurface of both the first and second ophthalmic lenses can have a dimension that is of, or less than, an optical wavelength.

In another embodiment, the operation of producing the first or the second ophthalmic lenses can further include associating a respective one of the first or second metasurface arrays with a non-solid substrate comprising a precursor form of any of the standardized lens bodies.

In another embodiment, the operation of associating the first or the second metasurface arrays with a respective one of the first or the second lens bodies can further include distributing the non-solid substrate using a molding process. The non-solid substrate can be curable to form the respective one of the first or the second ophthalmic lenses.

It will be appreciated that the differential optical properties can be one or more of the optical properties described herein. For example, the differential optical properties of the standardized lens bodies can include a reduced glare characteristic of the ophthalmic lenses. halo reduction, contrast enhancement, and/or aberration correction can also be tuned and customized for individual lens bodies of the group of standardized lens bodies, as described herein.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A depicts an embodiment of an ophthalmic lens having a first focal point;

FIG. 1B depicts an embodiment of the ophthalmic lens having a metasurface array inducing a second focal point;

FIG. 1C depicts an embodiment of the ophthalmic lens having the metasurface array on another side of a lens body and inducing the second focal point;

FIG. 1D depicts metasurface building elements of an ophthalmic lens of the present disclosure;

FIG. 2A depicts a standardized lens body having a first focal point;

FIG. 2B depicts the standardized lens body having a second focal point;

FIG. 2C depicts the standardized lens body having a third focal point;

FIG. 3A depicts a first lens body having a predetermined focal point;

FIG. 3B depicts a second lens body having the predetermined focal point of FIG. 3A;

FIG. 3C depicts a third lens body having the predetermined focal point of FIG. 3A;

FIG. 4A depicts a sample intraocular lens including a metasurface array;

FIG. 4B depicts an illustrative cross-section of the intraocular lens of FIG. 4A;

FIG. 4C depicts a cross-sectional view of one embodiment of an intraocular lens directing light into an eye, according to the principles of the present disclosure;

FIG. 4D depicts a cross-sectional view of one embodiment of an intraocular lens directing light into an eye, according to the principles of the present disclosure;

FIG. 4E depicts a cross-sectional view of one embodiment of an intraocular lens directing light into an eye, according to the principles of the present disclosure;

FIG. 4F is a partial cross-sectional perspective view of one embodiment of an ocular lens with feature for directing the light off axis toward a peripheral region of the retina, according to the principles of the present disclosure;

FIG. 5A depicts another intraocular lens having metasurface array in a predetermined arrangement;

FIG. 5B depicts the intraocular lens of FIG. 5A in a folded configuration for association with an eye during surgery;

FIG. 5C depicts the intraocular lens of FIG. 5A in an installed configuration with the eye and substantially having the predetermined configuration of a metasurface array of FIG. 5A;

FIG. 6A depicts a sample contact lens having a metasurface array in a predetermined arrangement;

FIG. 6B depicts the contact lens of FIG. 6A in a manipulated configuration for external association with the eye;

FIG. 6C depicts the contact lens of FIG. 6A in an externally installed configuration with the eye and substantially having the predetermined configuration of a metasurface array of FIG. 6A;

FIG. 7A depicts a side view of a patient having a modified field of view;

FIG. 7B depicts a top view of the patient of FIG. 7B having a modified field of view;

FIG. 8A depicts an operation of associating a metasurface array with a lens body using a molding process;

FIG. 8B depicts another operation of associating a metasurface array with a lens body using a molding process;

FIG. 8C depicts another operation of associating a metasurface array with a lens body using a molding process;

FIG. 8D depicts another operation of associating a metasurface array with a lens body using a molding process;

FIG. 9A depicts an operation of associating a metasurface array with a lens body;

FIG. 9B depicts another operation of associating a metasurface array with a lens body;

FIG. 10A depicts an operation of forming a metasurface array on a lens body;

FIG. 10B depicts another operation of forming a metasurface array on a lens body;

FIG. 11 depicts a flow diagram for manufacturing an ophthalmic lens; and

FIG. 12 depicts a flow diagram for manufacturing standardized ophthalmic lenses.

The use of cross-hatching or shading in the accompanying figures is generally provided to clarify the boundaries between adjacent elements and to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

The description that follows includes sample systems, methods, and apparatuses that embody various elements of the present disclosure. However, it should be understood that the described disclosure may be practiced in a variety of forms in addition to those described herein.

The present disclosure describes systems, devices, and techniques related to ophthalmic lenses (also referred to herein as “ophthalmic devices” or variations thereof). The ophthalmic lenses can have a metasurface array that defines, includes, or is otherwise associated with metasurface features of the ophthalmic lens. The metasurface features can be specifically tuned to modify an optical characteristic of the lens, without necessarily relying on the geometry of the lens to produce the desired optical effect. For example, the metasurface features can be configured across a body of the lens to reduce a glare characteristic of the lens, including contributing to halo reduction and/or contrast enhancement, among other optical characteristics described herein. The embodiments of the present disclosure thus go beyond traditional techniques for providing vision correction or other therapeutic purposes, for example, by changing the optical property of the lens using the metasurface features. Traditional techniques can limit lens design to overly bulky structures or to designs that limit the adaptability of the lens. This can lead to larger incisions and discomfort, as may be the case with intraocular lenses, as one example. These traditional techniques can also limit light, and thus reduce sensitivity of the lens, among other concerns.

The ophthalmic lenses of the present disclosure can mitigate such hindrances, thereby allowing for lenses that can be designed free from at least some geometric limitations. This can expand the ability to correct and modify optical properties across a greater a range of lens geometries, including expanding beyond previous techniques in order to reduce glare and correct other optical characteristics across a wider variety of lens sizes. To illustrate, the ophthalmic lenses can provide a metasurface array on a lens body. The metasurface array operates to modify an optical characteristic of the lens body, lens, or device more generally, and can be adaptable for the geometry of the lens, whether the lens is standardized or customized for certain therapeutic purposes. For example, the metasurface array can be configured to modify characteristics of light propagating through the lens body. This can include using a metasurface array to produce electric resonance effects, magnetic resonance effects and/or other appropriate effects in order to induce various changes in one or more optical properties associated with the lens. For example, in some cases, the metasurface array can employ the Pancharatnam-Berry phase for the modification of light described herein. In other cases, other techniques can be used to shift a phase of incident light, as contemplated herein. This can be facilitated by dimensioning the metasurface building elements of, or less than, an optical wavelength, such as being 400 nm or smaller, among other possible dimensions. The metasurface building elements can also be arranged in a predetermined configuration within the metasurface array and/or on the lens body to produce a desired optical effect, including having certain shapes, sizes, orientations, groupings, densities, patterns and so on. By way of example, the shape, size, orientation, group, density and/or pattern of features of the metasurface array can be tuned to reduce a glare characteristic of the lens, among other optical effects.

In one embodiment, the metasurface building elements can be defined by nano-posts. The nano-posts can be or be formed from amorphous silicon nano-posts. It will be appreciated that other materials can be used. The nano-posts can be arranged in a matrix material, such as being arranged in a polydimethylsiloxane (PDMS) substrate. The substrate can help hold or position the nano-posts in a desired orientation. The substrate can also facilitate depositing the nano-posts on a target surface (e.g., a lens body) in the desired orientation. As explained herein, this can allow the metasurface array to be used with a wide variety of lens surfaces and configurations, including substantially rotationally symmetric lenses, rotationally asymmetric lenses, and variations thereof. Such adaptability can also allow the metasurface array to be used with different lens types, including intraocular lenses and contact lenses, such as rigid gas permeable lenses and/or scleral lenses, as a few examples. It will be appreciated, however, the example lenses are described for purposes of illustration, and that the ophthalmic lenses described herein can be used in a wide variety of contexts. For example, in additional embodiments, the ophthalmic lenses can be a hybrid lens, including a lens having a soft periphery. Additionally or alternatively, the lenses can include a hydrogel component. As further examples, the ophthalmic lens can have applications in various intra corneal lens, corneal inlay, corneal onlay, and implantable contact lens contexts, as contemplated herein. In other cases, other applications are possible.

The ophthalmic lens of the present disclosure can be subject to physical manipulation during use and installation. The metasurface array described herein allows the target optical characteristics to be maintained after such physical manipulation. The ophthalmic lens thus exhibits a durability consistent with the target use of the lens, with the metasurface features being sufficiently robust to withstand the target use. For example, the ophthalmic lens can be a foldable lens that can be folded, rolled, or otherwise physically handled, such as may be accomplished during association with an eye during surgery. The metasurface array can withstand this physical handling continues to appropriately modify the target optical characteristics after the handling ceases.

With this durability, the ophthalmic device can be adaptable to a wide variety of intraocular lens contexts. Intraocular lenses can be surgically associated with a user's eye for permanent or semi-permanent use. This often involves creating an incision in the eye and inserting a folded intraocular lens through the incision for introduction to the installation location in the eye. In this regard, the ophthalmic lens described herein can be folded for insertion through such an incision. And when unfolded and associated with the installation location of the eye, the ophthalmic lens can maintain or otherwise exhibit the desired optical effect induced by the metasurface feature.

The ophthalmic devices of the present disclosure can also be tailored for use as intraocular lenses because the metasurface array facilitates manufacturing a lens body that is substantially free from geometric considerations. The lens body can also be constructed in order to be folded or rolled in a manner to fit through an incision that is substantially smaller than traditional approaches, thus reducing the risk of complications. For example, the device can be folded or rolled to fit an incision of between about 1 mm and 2 mm, including in some cases being able to fit through an incisions of less than 1 mm. For example, the lens body of the present disclosure, as a non-limiting example, could be substantially flat and have a 0.25 mm thickness. It will be appreciated that the thickness of the lens body and lens more generally can vary along one or more dimension of the lens. In this regard, to the extent that the lens body has a portion with a thickness of about 0.25 mm, this is not necessarily a uniform thickness. For example, an optical zone can have a thickness different from a peripheral zone of the lens. The thickness of the lens can facilitate folding the lens. In some cases, the diameter of the lens can also facilitate folding the lens for introduction into the incision, such as can be the case where the lens exhibits a 6 mm diameter. In contrast, traditional intraocular lenses can require larger incisions for installation. In addition, large incision sites can increase the risk of complications during surgery, such as infection.

To facilitate the foregoing ophthalmic lens designs and functions, a variety of manufacturing techniques are disclosed herein. Broadly, the manufacturing techniques can allow for a standardized substrate that defines or forms a portion of a lens body. This can substantially reduce manufacturing cost and facilitate the incorporation of an expansive variety of lens parameters in the device. For example, because the optical characteristic, such as characteristics associated with vision correction, are tuned via the metasurface array, the geometry of the lens shape can be substantially standardized across a spectrum of ophthalmic devices having different optical characteristics. Conversely, a range of different lens geometries can have similar optical characteristics by tuning the metasurface array accordingly.

In one embodiment, the ophthalmic lens of the present disclosure can be produced using a molding process. For example, a first mold portion of a molding apparatus can be configured to receive the metasurface array. As described herein, this array can include an arrangement of metasurface building elements, such as silicon nano-posts, arranged in a predetermined configuration. A lens material, such as a liquid lens material, can be substantially applied to the metasurface array within the molding apparatus, coupling the metasurface array with a non-solid substrate. A second mold portion of the molding apparatus can be used to form a lens shape from the liquid lens material and the metasurface array. For example, the second mold portion can advance toward the first mold portion to distribute the liquid lens material over the metasurface array, conforming each into a mold shape substantially defined by the first and second mold portions. A curing process can be used to form the final lens body associated with the metasurface array.

In this regard, the molding process can be tuned to produce a standardized lens body geometry that is substantially defined by the molding apparatus. But while the geometry is standardized, the optical properties for each manufactured lens can be different, for example, based on the arrangement of the metasurface array. Without reconfiguring machine tooling and other parameters traditionally associated with manufacturing different lens geometries, manufacturing costs can be reduced. The process can also be adapted to ultra-fine adjustments of the optical parameters using the metasurface array with the geometry of the lens body being relatively constant.

It will be appreciated that other manufacturing methods are possible, and are contemplated herein, including methods which are used to produce lens bodies of different sizes and geometries. For example, the foregoing molding process can implement molds of different sizes and configurations, as may be desired for different ophthalmic lens types, such as intraocular lenses, contact lenses, and so on, including adjusting the mold or mold set-up for treating different conditions, including for treating eyes with asymmetrical contours. Other manufacturing techniques can use a lathe process to form some or all of the lens body, which is subsequently associated with a metasurface array. For example, the metasurface array can be manufactured separately from the lens body and form a peelable sheet or other structure that is subsequently associated with the lens body. In other cases, the metasurface array can be formed more directly on a surface of a lens body, for example, through a dry etching or lithography process. In other cases, other techniques are possible and described herein.

Reference will now be made to the accompanying drawings, which assist in illustrating various features of the present disclosure. The following description is presented for purposes of illustration and description. Furthermore, the description is not intended to limit the inventive aspects to the forms disclosed herein. Consequently, variations and modifications commensurate with the following teachings, and skill and knowledge of the relevant art, are within the scope of the present inventive aspects.

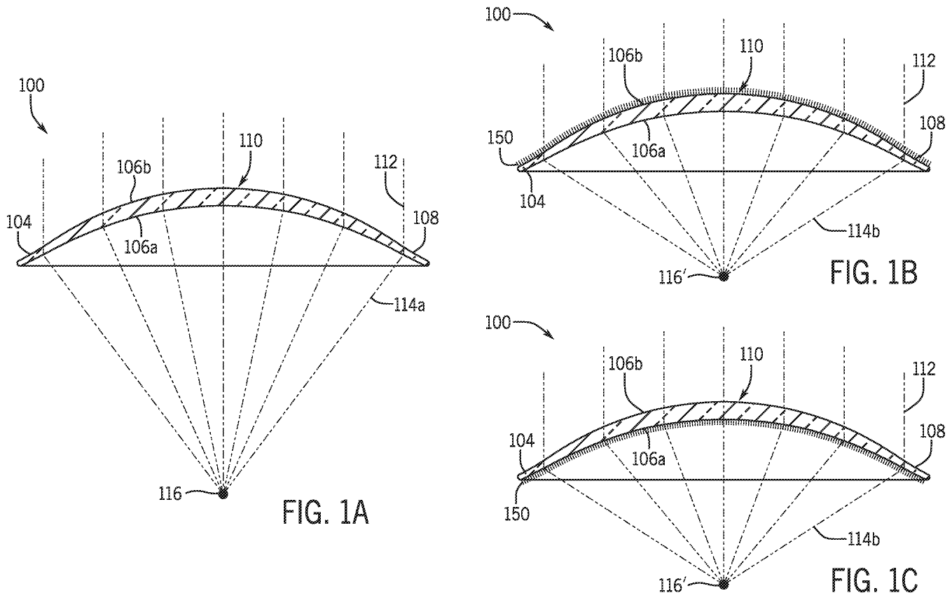

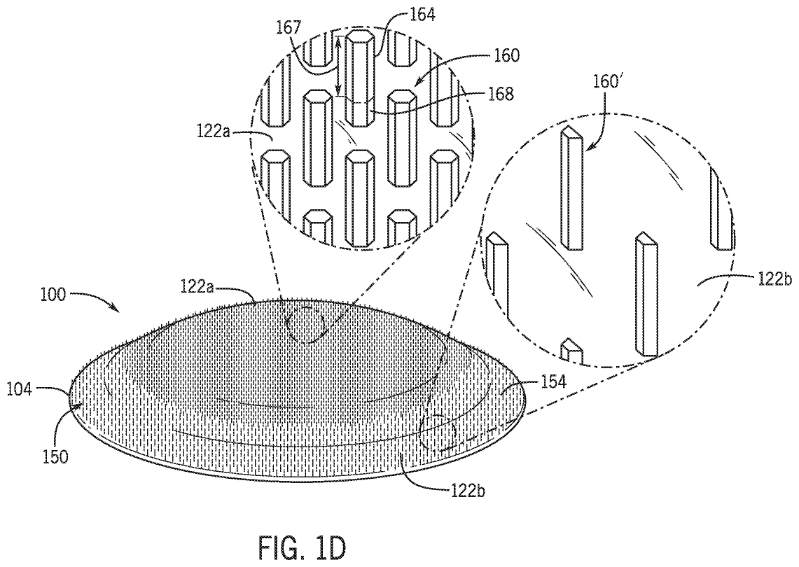

FIG. 1A depicts an ophthalmic lens 100. The ophthalmic lens 100 includes a lens body 104 having a posterior surface 106a and an anterior surface 106b. The lens body 104 can be constructed to have a variety of different optical and structural properties, as described herein. In this regard, the lens body 104 can have an optical region 110 through which light traverses and produces a given optical effect. For example, from the anterior surface 112 106, light can generally propagate along a path 112, and from the posterior surface 106a, light can propagate along a path 114a. The path 114a can generally converge toward a focal point 116 that is separated from the posterior surface 106a by a predefined distance. The lens body 104 can also have various other structural features which can be adapted to provide structural stability to the lens, such as adapting the lens for appropriate landing on an eye. This can be generally defined by a tangential region 108, which can substantially encircle the optical region 110.

The ophthalmic lens 100 of FIG. 1A is shown without metasurface features or other elements that would otherwise impact the arrangement of light such as a metasurface array. In this manner, the paths 112, 114a can be substantially determined based on a geometry of the lens body 104. For example, the convex contour of the anterior surface 106b can cause light to transition from and between the path 112 and the path 114a as the light traverses the body 104. FIG. 1 thus shows traditional techniques where the geometry of lens body 104 would be tuned in order to modify an optical property of the lens, such as the focal point.

The embodiments described herein allow optical properties of a lens to be modified without necessarily relying or tuning the geometric properties of the underlying lens structure. Metasurface features can be associated with the lens body 104, for example, using a metasurface array, in order to modify the properties of the ophthalmic lens 100.

In this regard, FIG. 1B depicts an embodiment of the ophthalmic lens 100 having a metasurface array 150 associated with the lens body 104. With the association of the metasurface array 150, light is propagated along a new path from the posterior side 106a, substantially converging at a focal point 116′. As explained in greater detail below, the metasurface array 150 can include, define, or be a metasurface or constitute the metasurface features that operate to induce light along a new direction or path. FIG. 1B shows enlarged and illustrative metasurface features for purposes of illustration. The lens body 104 of FIGS. 1A and 1B can be of identical construction, such as being formed from a group of standardized lens bodies, and the focal point 116′ of FIG. 1B being different from the focal point 116 of FIG. 1A due to the introduction of the metasurface array 150. For purposes of illustration, the metasurface array 150 is shown associated with the anterior surface 106b. In other cases, such as that shown in FIG. 1C, the metasurface array 150 can be associated with the posterior surface 106a. Associating the metasurface array 150 with the posterior surface 106a can also induce the focal point 116′, as shown in FIG. 1C.

It will be appreciated that FIGS. 1A-1C show the change in the focal point induced by the introduction of the metasurface array 150. In other cases, other optical characteristics can optionally be modified or maintained, as facilitated by the metasurface array 150. Sample optical characteristics can include aberrations characteristic of the lens body, which can be lowered by the introduction of the metasurface array 150. For example, the metasurface array 150 can be adapted to facilitate correction of chromatic aberration, monochromatic aberration, and so on. As another example, the optical characteristic can include a glare characteristic of the lens body, which can be lowered by the metasurface array. For example, the glare characteristic can be measured using the Unified Glare Rating, as one example. Classifications on the Unified Glare Rating (UGR) typically range from 5 to 40. A lower number corresponds to a lower glare. The UGR may be reduced using the metasurface arrays described herein. For the sake of illustration, a UGR value may be reduced by as much as 5%, by as much as 10%, by as much as 15%, or more in a given illumination environment, using the ophthalmic devices described herein. In other cases, other optical properties can be modified or maintained, including those associated with vision correction, disease treatment, therapeutic uses, cosmetic functions, and so on, including use in treating colorblindness

While it will be appreciated that many combinative optical properties and effects can be achieved, in one embodiment, the optical property can include one or more focal points of the lens body. In this regard, the metasurface array can operate to induce optical properties associated with the bifocal, progressive multifocal and trifocal for vision correction.

As another example, the optical property can include an astigmatism correcting property. In this regard, the metasurface array can operate to define or modify focal distances relative to respective meridians of the lens, such as having a first focal distance associated with a first meridian and a second focal distance associated with a second meridian.

As another example, modifying the focal point can include modifying a decentralized focal point. In this regard, the metasurface array can operate to define the focal point as being decentralized relative a central axis of the lens. Additionally or alternatively, this can involve defining or modifying one or more focal points that focused at peripheral location disposed at a distance from fovea.

In another embodiments, metasurface features can combine with refraction and/or diffraction based optical zone. For example, the lens can include a central optic zone having metasurface structures, a peripheral optic zone surrounding central optic zone comprised by refraction and/or diffraction based optical property zone.

It will be appreciated that the lens body 104 can be any appropriate geometry, which may be adapted for a particular application. In some cases, the lens body 104 can have a standardized geometry to facilitate the efficient manufacture of substantially high volumes of lenses. Despite the geometry being standardized, the metasurface array 150 can be tuned to induce different optical effects in certain ones of the lenses, such as a reduction in a glare characteristic, a halo reduction, aberration correction, and so on. In other cases, the lens body 104 can have a geometry that is customized to particular patient. This can be the case for certain therapeutic uses of the lens, such as that where the lens is surgically associated with the eye and a custom fit is desired. In this regard, the metasurface array 150 can be tuned to produce a desired optical effect, notwithstanding the customized geometric shape of the lens. Wide angle lenses, such as those having a wide angle contact lens body, and other lens shapes can also be used as appropriate.

FIG. 1D illustrates a perspective view of the ophthalmic lens 100 having the lens body 104 and the metasurface array 150. FIG. 1D shows sample structures and compositions of the array 150 that can facilitate functions of the various ophthalmic devices described herein. It will be appreciated that the structures of the metasurface array are depicted for purposes of illustration, and include enlarged features meant to illustrate the present disclosure, rather than provide an indication of an actual scale of size.

The lens body 104 is shown as having a substantially rotationally symmetric profile, as can be used for various types of vision correction. In other cases, the lens body 104 can form a substantially rotationally asymmetric profile, irregular profile, and/or include substantially flat sections, as appropriate for a given application. In this manner, the anterior surface 106b of FIG. 1D is non-planar and curved in a manner that can be configured to match a geometry of an eye. The metasurface array 150 is adapted to be associated with this non-planar external layer of the lens body 104, as shown in FIG. 1D. In other cases, the metasurface array 150 can be associated with other surface of the lens body 104, including embodiments where some, but not necessarily all, of the lens body is associated with the array.

Broadly, the metasurface array 150 operates to modify an optical characteristic of the ophthalmic lens 100. As described above, this could include modifying a focal point of the lens from the focal point 116 of FIG. 1A to the focal point 116′ of FIG. 1B, among a wide spectrum of available optical property modifications. To facilitate the modification of such optical properties, the metasurface array 150 can include an arrangement of metasurface building elements 160, as shown in FIG. 1D.

While the metasurface building elements 160 can take many forms, the elements 160 are shown in FIG. 1D as including a collection of nano-posts. The nano-posts can be physical structures that are arranged in a predetermined manner relative to a surface of the lens body 104 in order to influence or modify light that propagates therethrough. For example, the nano-posts can be dimensioned smaller than a cycle wavelength of light, such as being about or smaller than 400 nm in certain cases. The dimensioning and arrangement of the nano-posts along the lens body surface can induce certain effects that change or modify light that impacts the posts, this can include electric resonance effects, magnetic resonance effect and/or other appropriate effects that operate to modify the light. For example, in some cases, the metasurface array can employ the Pancharatnam-Berry phase for the modification of light described herein. In other cases, other techniques can be used to shift a phase of incident light, as contemplated herein. For example, the collection of nano-posts can cooperate to modify an optical characteristic of the lens, such as modifying a focal point of the lens and/or other features that can optionally be associated with vision correction.

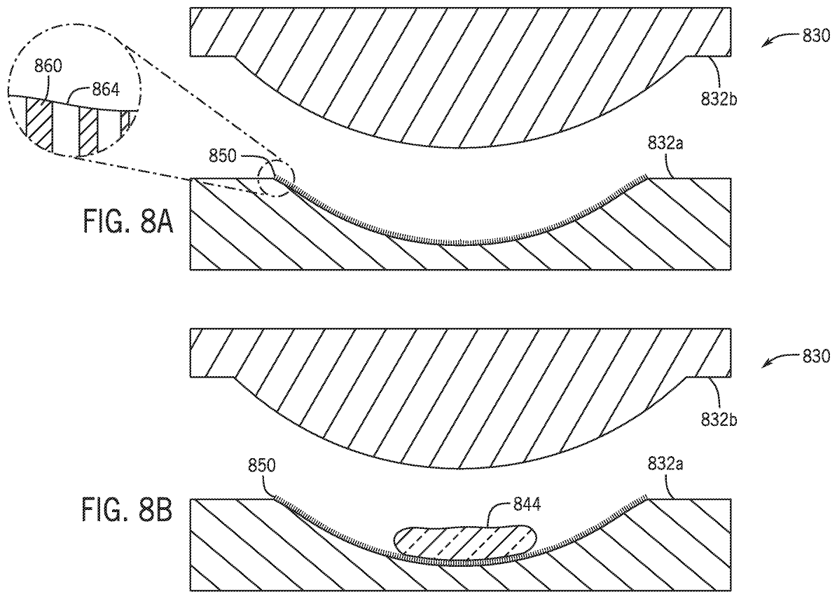

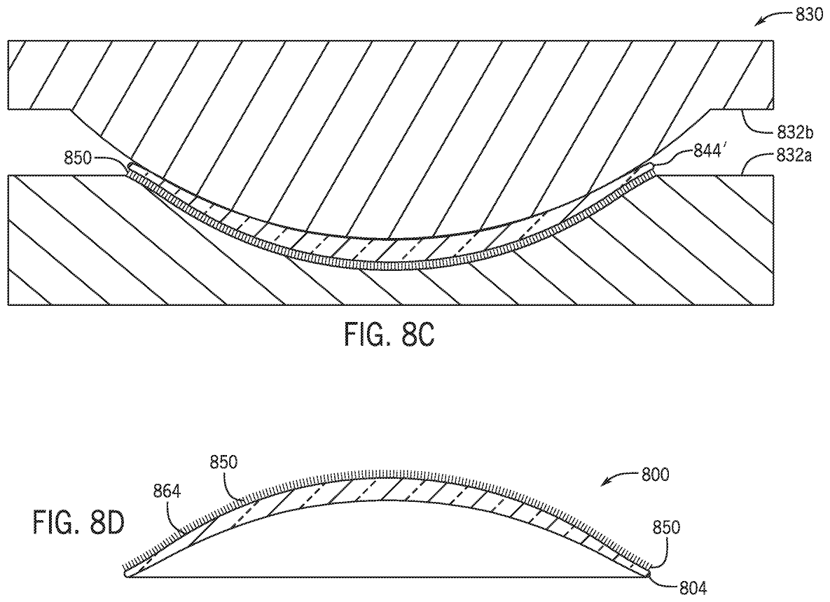

The nano-posts can be formed from various materials in order to generate a desired optical effect. For example, the nano-posts can be formed form an amorphous silicon. Additionally or alternatively, a titanium dioxide and/or a silver dioxide material can form some or all of the nano-posts. As described in greater detail below, the nano-posts can be formed from an etching process, including using lithography. In this manner, a starting material or substrate layer, such as a layer of amorphous silicon can be etched to form the collection of nano-posts in the desired shape. The collection of nano-posts can then be associated with the lens body in a variety of ways, including using a molding process and/or peelable sheets, as described below with respect to FIGS. 8A-10B. In other cases, other manufacturing techniques can be used and are contemplated herein.

In the sample of FIG. 1D, the metasurface array 150 is shown including a metasurface building element 160. The metasurface building element 160 can be one of numerous metasurface building elements arranged in a matrix 154 of the metasurface array 150. The matrix 154 can be used to associate the metasurface building elements, such the metasurface building element 160, with the lens body 104. For example, the matrix 154 can be, in certain embodiments, formed from a polydimethylsiloxane material and/or other materials, including polymers and flexible substrates. In some cases, the matrix 154 can have adhesive properties that allow the matrix 154 to bind or otherwise attach to the lens body 104. The matrix 154 can be used as a sacrificial matrix, where some or all of the matrix 154 is removed prior to the use of the ophthalmic lens 100 by a user. In other cases, the matrix 154 can remain fully intact during use of the ophthalmic device 100, including cases in which the matrix fully and/or at least partially encompasses the metasurface building elements to facilitate maintaining the desired orientation and arrangement for inducing the desired optical effect.

As described herein, the metasurface building elements can be defined by a collection of nano-posts. In the sample of FIG. 1D, the metasurface building element 160 is shown as being defined by a nano-post 164. The nano-post may have a shape, size, orientation and/or other characteristic or property in order to generate a desired optical effect. For example, the nano-post 164 can have a substantially hexagonal shape, as shown in FIG. 1D; however, this is not required. In other cases, other shapes are contemplated, including rectangular, circular and/or irregular or asymmetric shapes, among other possibilities. Such characteristics of the nano-post 164 can be tuned in order to induce the desired optical properties, as described herein.

In certain embodiments, at least a section of the nano-post 164 can be directly associated with the matrix 154. To illustrate, FIG. 1D shows a matrix portion 168 of the nano-post 164. The matrix portion 168 can be a section of the nano-post 164 that is encompassed by the matrix 154, thereby facilitating the association of the nano-post 164 with the lens body 104. In some cases, the matrix portion 168 can include all or substantially all of the nano-post 164, whereas in other cases, the matrix portion 168 can include a reduced amount or optionally be removed or located on a bottom surface of the nano-post 164, as appropriate for a given application.

In this regard, the nano-post 164 is arranged in a particular configuration in order to facilitate the optical effects desired herein. The nano-post 164 can generally maintain this configuration through physical manipulation of the ophthalmic lens, such as the manipulation of the lens during surgery (e.g., for intraocular lens embodiment) and/or external use in a contact lens environment, using the matrix 154 and/or other structure, substrate, or method. To illustrate, FIG. 1D shows the metasurface building element 160 including a nano-post 164 in a sustainably vertical configuration relative to a surface of the lens body 104 and hexagonal in shape. FIG. 1D also shows the nano-post having a post height 167, which is so dimensioned from an optical wavelength, including being smaller than a cycle wavelength of light. In this regard, the post height 167 can be 400 nm or smaller. In other cases, the post-height 167 can be larger than 400 nm, such as being around 500 nm, 900 nm or greater, as appropriate for a desired optical property. It will be appreciated that the height 167 is presented as one sample dimension. Additionally or alternatively, the nano-post 164 can have other dimensions of, or smaller than, cycle wavelength of light, including a width, thickness, and so on.

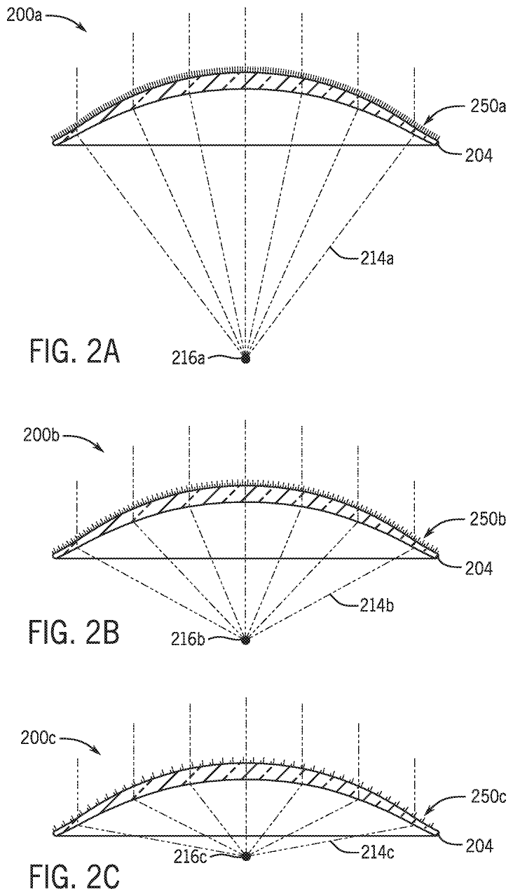

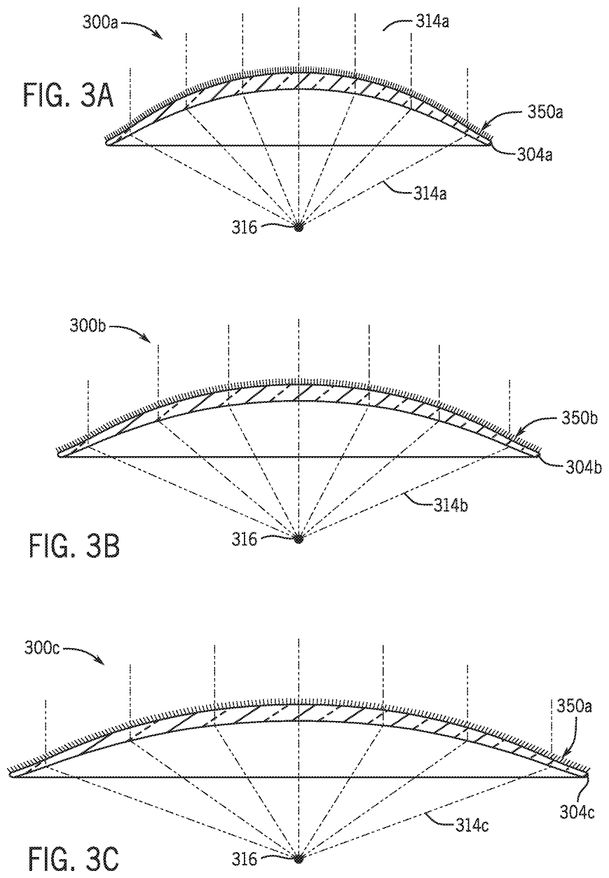

In view of the physical characteristics of the metasurface building element 160, outlined above, the behavior of light through the lens body 104 can be modified. For example, and as shown in greater detail in FIGS. 2A-2C below, the physical characteristics of the metasurface can be used to change a focal point of the lens. The cumulative optical effect of the lens can be influenced by metasurface features of the metasurface array 150. For example, the optical property, such as the focal length, can be modified for the overall lens based on the arrangement of the collection of metasurface building elements, including the particular configuration and physical characteristics of the metasurface building elements. For example, the collection of metasurface building elements of the metasurface array 150 can include individual metasurface building elements, including nano-posts of dissimilar shapes, dissimilar heights, orientations, and so on. Such differences can allow for the ultra-fine tuning of the lens's optical properties, as well as adjusting for the unique geometry of lens, as described in greater below with respect to FIGS. 3A-3B.

To illustrate, FIG. 1D shows a second metasurface building element 160′. The second metasurface building element 160′ can have a different orientation, size, and shape, as examples, than that of metasurface building element 160. The different shape of the second metasurface building element 160′ can thus influence light in a manner that is different than the metasurface building element 160. This may be desirable, for example, where the second metasurface building element 160′ is arranged at geometrically distinct portions of the lens body 104 than the metasurface building element 160, and thus interacts with light in a distinct manner. Further, it may be desirable to have light interact with metasurface building elements in different manners to realize the combinative optical effects of light interaction with the metasurface building element 160 and the second metasurface building element 160′, including the combinative effects that can be obtained with glare and aberration reduction. Cosmetic applications, including influencing colors, are also contemplated.

FIG. 1D also illustrates that in addition to the physical characteristics of the metasurface building elements outlined above, the density and grouping of the metasurface building elements can also be modified along the surface of the lens body 104. For example, it may be desirable to have a higher concentration of metasurface building elements at one area of a lens, and a lower concentration of metasurface building elements at another area of a lens. This differential can, for example, account for the curvature of the lens, where a curved lens is used. Additionally or alternatively, it can contribute to the modification of the various optical effects described herein.

In this regard, FIG. 1D shows the metasurface array including a first portion 122a having a first density of metasurface building elements. FIG. 1D also shows the metasurface array 150 having a second portion 122b having a second density of metasurface building elements. In some cases, the metasurface building elements can define a gradient between the density of portion 122a and portion 122b. In other cases, the change in density between the portions 122a, 122b can be abrupt or discontinuous, including having portions of the metasurface array substantially free from metasurface building elements, as appropriate for a given application.

In this regard, it will be appreciated that the collection of nano-posts, or any of the metasurface building elements described herein can be used to induce combinative optical effects with the geometry of the lens. For example, the lens body 104 may have a geometry that exhibits certain optical properties associated with light diffraction and/or refraction. The collection of nano-posts can thus operate to influence the characteristics of light through the lens body that are induced by the diffraction and/or refraction associated with the lens body 104. This can be beneficial, for example, where the geometry of the lens body is used to provide a certain therapeutic effect, including geometries allowing for a particular fitting of the lens to a patient's eye.

The metasurface array and embodiments herein can be used to induce various different optical properties across lenses having geometrically same or similar lens bodies. For example, the metasurface array can employ metasurface features to induce optical changes, rather than rely solely from the geometric shape of the lens body. To illustrate the foregoing, FIGS. 2A-2C show a series of ophthalmic lenses of a common or standardized lens body with each having different optical properties, such as each having a different focal point. For example, a metasurface array, such as those discussed above can be different and tuned as to each of the individual ophthalmic devices in order to induce the different optical effects for devices having the same of similar lens geometry.

With reference to FIG. 2A, an ophthalmic lens 200a is shown. The ophthalmic lens 200a has a lens body 204 and associated with a metasurface array 250a. The metasurface array 250a can include an arrangement of metasurface building elements in order to facilitate the ophthalmic lens 200a directing light along a path 214a toward a focal point 216a.

With reference to FIG. 2B, an ophthalmic lens 200b is shown. The ophthalmic lens 200b has the lens body 204, which can be similar or identical to the lens body 204 of FIG. 2A. The lens body 204 of FIG. 2B is associated with a metasurface array 250b, which can be different than the metasurface array 250a. For example, the metasurface array 250b can include metasurface building elements of different size, shape, orientation, density and so on, as compared to the metasurface array 250a. In this manner, the metasurface array 250b can operate to induce different optical characteristics for the ophthalmic lens 200b, despite the lens body 204 being geometrically the same or similar to that of the ophthalmic lens 200a. As shown in FIG. 2B, the metasurface building elements of the metasurface array 250b can be arranged in order to facilitate the ophthalmic lens 200b, directing light along a path 214b toward a focal point 216b, that is different than the focal point 216a.

With reference to FIG. 2C, an ophthalmic lens 200c is shown. The ophthalmic lens 200c has the lens body 204, which can be similar or identical to the lens body 204 of FIG. 2A or 2B. The lens body 204 of FIG. 2C is associated with a metasurface array 250c, which can be different than the metasurface array 250a and/or 250b. For example, the metasurface array 250c can include metasurface building elements of different size, shape, orientation, density and so on, as compared to the metasurface array 250a and/or 250b. In this manner, the metasurface array 250c can operate to induce different optical characteristics for the ophthalmic lens 200c, despite the lens body 204 being geometrically the same or similar to that of the ophthalmic lens 200a and/or 200b. As shown in FIG. 2C, the metasurface building elements of the metasurface array 250c can be arranged in order to facilitate the ophthalmic lens 200c, directing light along a path 214c toward a focal point 216c, that is different than the focal points 216a, 216b.

Accordingly, the lens body 204 can be produced from a standardized process, such as that illustrated in FIGS. 8A-10B. For example, the lens body 204 can be one of a group of standardized lens shapes. This can reduce manufacturing complexity, allowing the optical characteristics of resulting ophthalmic lenses to be substantially defined by the metasurface array, rather than the geometric properties of the lens body. It will be appreciated that FIGS. 2A-2C show the change in focal point as an illustrative optical property that can be modified as a result of the metasurface array. In other cases, more or different optical properties can be modified, including those associated with aberrations, glare, vision correction, and so on.

The metasurface array and embodiments herein can also be used to induce substantially the same optical properties for lenses having disparate geometries. For example, the metasurface array can employ metasurface features that induce optical changes, rather than rely solely from the geometric shape to induce optical effects. In this manner, the metasurface features can be tuned to account for the geometric shape of the lens body, in order to influence light traversing the lens body to exhibit a common optical property. This can be beneficial, for example, where lenses of different sizes and shapes, such as those that are used to treat various different conditions, each have a common focal point or other commonly desired optical property across the different lens types. To illustrate the foregoing, FIGS. 3A-3C shows a series of ophthalmic lenses, each having substantially the same focal point, but with geometrically dissimilar lens bodies. For example, a metasurface array, such as those discussed above, can be different and tuned as to each of the individual ophthalmic devices in order to induce a common optical effect across devices having the different lens geometry.

With reference to FIG. 3A, an ophthalmic lens 300a is shown. The ophthalmic lens 300a has a lens body 304a and is associated with a metasurface array 350a. The metasurface array 350a can include arrangement of metasurface building elements in order to facilitate the ophthalmic lens 300a directing light along a path 314a toward a focal point 316.

With reference to FIG. 3B, an ophthalmic lens 300b is shown. The ophthalmic lens 300b has a lens body 304b, which can be geometrically different than the lens body 304a of FIG. 3A. The lens body 304b of FIG. 3B is associated with a metasurface array 350b, which can be different than the metasurface array 350a. For example, the metasurface array 350b can include metasurface building elements of different size, shape, orientation, density and so on, as compared to the metasurface array 350a. In this manner, the metasurface array 350b can operate to induce a common optical characteristic for the ophthalmic lens 300b (e.g., focal point 316), despite the lens body 304b being geometrically different to that of the ophthalmic lens 300a. As shown in FIG. 3B, the metasurface building elements of the metasurface array 350b can be arranged in order to facilitate the ophthalmic lens 300b directing light along a path 314b toward the focal point 316, which is the same or substantially similar to the focal point 316 of the ophthalmic lens 300a of FIG. 3A.

With reference to FIG. 3C, an ophthalmic lens 300c is shown. The ophthalmic lens 300c has a lens body 304c, which can be geometrically different than the lens body 304a of FIG. 3A and/or the lens body 304b of FIG. 3B. The lens body 304c of FIG. 3C is associated with a metasurface array 350c, which can be different than the metasurface array 350a and/or 350b. For example, the metasurface array 350c can include metasurface building elements of different size, shape, orientation, density and so on, as compared to the metasurface array 350a and/or 350b. In this manner, the metasurface array 350c can operate to induce a common optical characteristic for the ophthalmic lens 300c (e.g., focal point 316), despite the lens body 304c being geometrically different to that of the ophthalmic lenses 300a, 300b. As shown in FIG. 3C, the metasurface building elements of the metasurface array 350c can be arranged in order to facilitate the ophthalmic lens 300c, directing light along a path 314c toward the focal point 316, which is the same or substantially similar to the focal point 316 of the ophthalmic lens 300a of FIG. 3A and the ophthalmic lens 300b of FIG. 3B.

It will be appreciated that FIGS. 3A-3C show the change in focal point as an illustrative optical property that can be modified as a result of the metasurface array. In other cases, more or different optical properties can be modified, including those associated with aberrations, glare, vision correction, and so on.

The metasurface arrays described herein can be used in a wide variety of applications, including applications where the lens is configured for installation during surgical producers or otherwise installed by a medical practitioner. As one example, the metasurface array can be used in a lens or lens system that comprises or defines an intraocular device or lens. The intraocular lens can be used to treat cataracts or myopia, and is thus typically associated with an eye during a surgical procedure. The metasurface array used with the intraocular lens can allow the lens body to exhibit a variety of different physical characteristics, for example, because the modification of light and optical characteristics can be controlled by the metasurface array rather than solely by the geometry of the lens body. In this regard, the intraocular lens can be substantially flat in a pre and post-surgical configuration, and allow the lens body to have certain other characteristics that can reduce the incision size during the surgical procedure, including having a thickness of 0.25 mm or less, and being capable of folding and/or rolling, and insertion through incision of 2 mm or less, such as an incision of 1 mm or less.

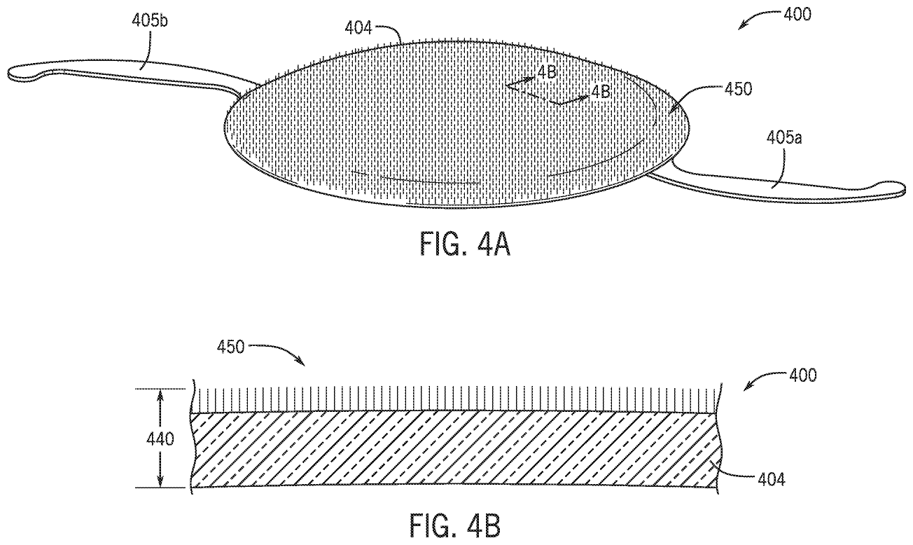

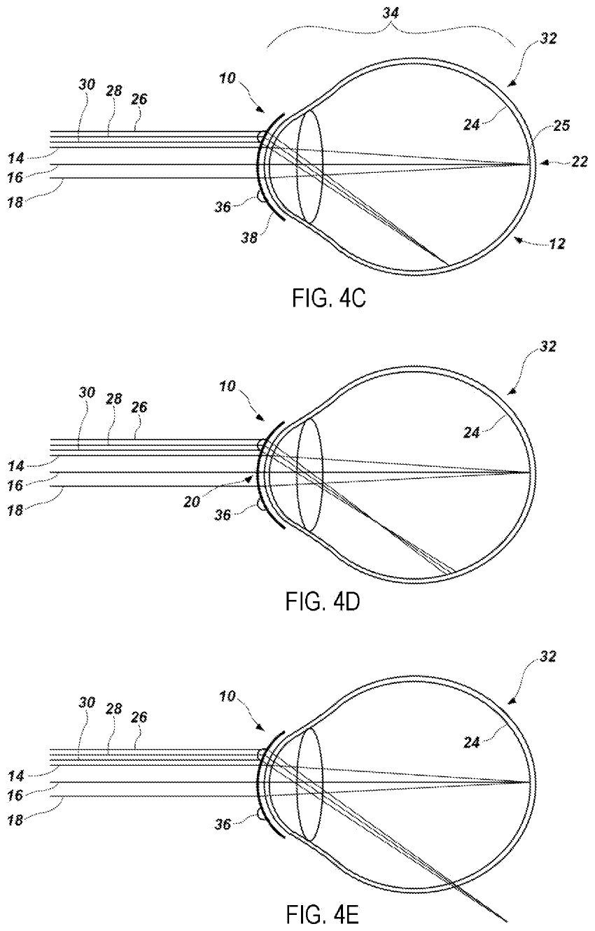

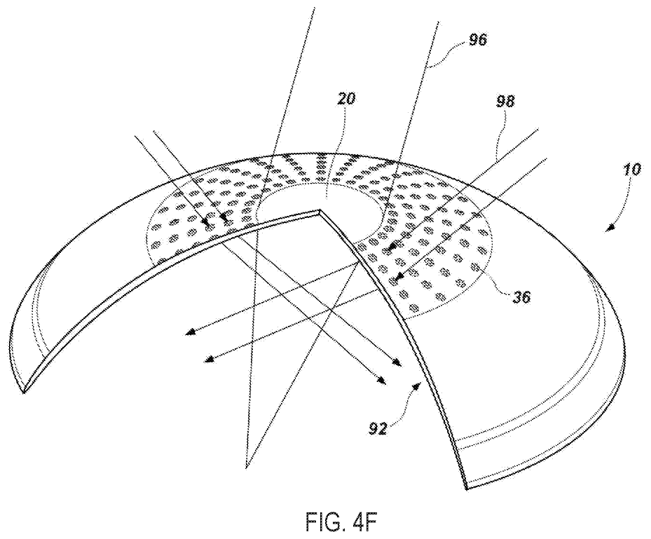

In this regard, FIGS. 4A-4B depict an ophthalmic lens 400. The ophthalmic lens 400 can be an intraocular lens or device having a metasurface array. In this regard, FIG. 4A shows the ophthalmic lens having a lens body 404 and a metasurface array 450. The metasurface array 450 can be substantially analogous to the metasurface arrays described herein; redundant explanation of which is omitted here for clarity.

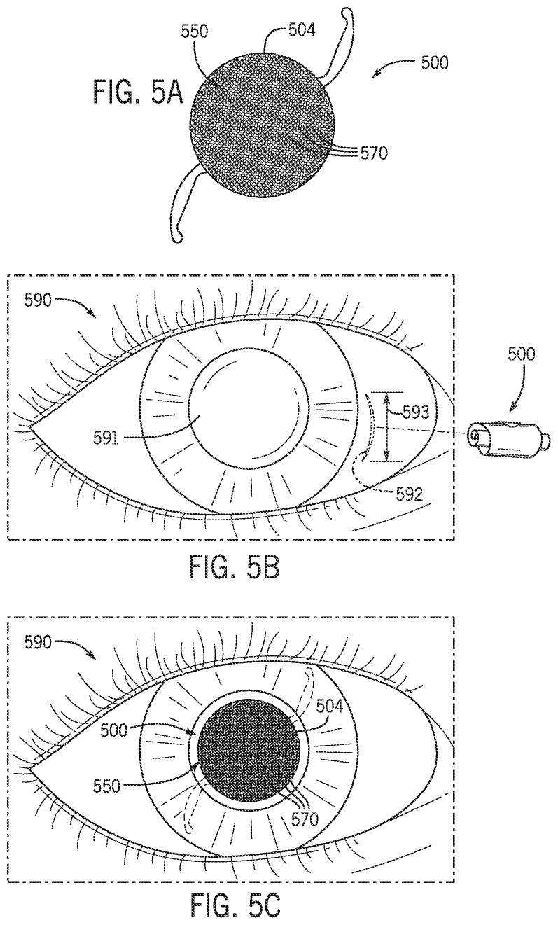

Notwithstanding the foregoing, the metasurface array 450 can be adapted for use with the intraocular lens. This can involve manufacturing the metasurface array 450 and associated lens body 404, according to the manufacturing techniques herein. For example, the metasurface array 450 can be sufficiently durable to maintain the target optical properties and modification subsequent to a surgery process for installing the lens. In some embodiments and as show in greater detail with respect to FIGS. 5A-5C, the arrangement of metasurface building elements of the metasurface array can be maintained through and subsequent to a surgical procedure.

The ophthalmic lens of FIG. 4A also includes other adaptions for intraocular lens applications. For example, the lens body 404 can be configured to have certain features that facilitate alignment of the lens during surgery. FIG. 4A shows the lens body 404 having a first haptic feature 405a and a second haptic feature 405b. The first and second haptic features 405a, 405b can facilitate aligning the lens body 404 with certain features of an eye during surgery. It will be appreciated that in other cases, other haptic features can optionally be used, including those which define wing-type shapes and other shapes for aligning and/or structurally landing the lens 400 relative to an eye.

With reference to FIG. 4B, an illustrative cross-section of the ophthalmic lens 400 of FIG. 4A is shown. FIG. 4B shows that that ophthalmic lens 400 can generally have a substantially flat shape, such as that prior to installation during surgery. In this regard, FIG. 4B shows the ophthalmic device having a thickness 440. The thickness 440 can be generally about 0.25 mm. So dimensioning the ophthalmic lens 400 can allow the lens to be folded, rolled, or otherwise physically manipulated during surgery in a manner that allows the ophthalmic lens 400 to fit through a substantially small incision, as shown in FIGS. 5A-5C. The thickness 440 can also be tailored to induce a certain rigidity for the ophthalmic lens 400, allowing the ophthalmic lens to retain its shape after being manipulated for use. In this regard, it will be appreciated that the 0.25 mm is one sample dimension, which can further be adapted based on the material of the ophthalmic lens. As such, in some cases, the thickness 440 can be less than 0.25 mm, such as being less than 0.20 mm, whereas in other cases the thickness 440 can be larger, such as being less than 0.50 mm or less than 1 mm.

In addition, according to one exemplary embodiment, a metasurface array can be incorporated onto a surface of a contact lens to treat myopia progression, particularly in young people, as illustrated in FIGS. 4C-4F. Myopia is caused by an undesirable axial length of the eye. It has been found that if the growth of the eye's axial length can be controlled during a child's youth, myopia or hyperopia can be reduced or even eliminated in the child's adulthood years.

The growth of the eye's axial length can be affected by visual feedback received in the retina. The visual feedback can be used to balance the axial length of the eye with the collective focusing ability of the cornea and crystalline lens. The eye uses the focal point of the light focused on the retina to determine when the eye's axial length is balanced. Such visual feedback may be based on the entire surface area of the retina, and not just the central portions of the retina dedicated to central vision. Thus, if the periphery of the retina, which has a greater surface area than the central region, receives visual feedback to extend the axial length, the eye may respond by growing to increase the axial length of the eye. This may occur in cases where the central vision is already balanced. Thus, such visual feedback can cause the central vision to become out of focus.

The light directed towards the peripheral regions of the retina can provide a stimulus that the eye can interpret as visual feedback to determine a rate of growth for the eye. In some examples, the light directed towards the peripheral regions of the retina is focused exactly on the peripheral regions of the retina. By causing the focal point of the peripherally directed light to be exactly on the retina, the eye may alter the growth rate of the eye so that the axial length of the eye maintains a consistent balance with the eye's focusing power. This may cause the eye to grow slower or stop growing altogether.

In other examples, the light may be focused short of the peripheral regions of the retina. As a result, the focal point of the directed light is in front of the retina. Such a stimulus may cause the eye to have peripheral myopia. This may have the effect of causing the eye to slow growth or stop growing altogether.

Generally, young children begin with a hyperopic condition where the focal point is formed behind the retina. Thus, the eye has an early stimulus to cause the eye to grow in a manner to correct the balance between the eye's focusing power and axial length. In cases where a child has a central hyperopic condition, light can be directed to the peripheral regions of the retina to be purposefully focused behind the retina. This may provide an additional stimulus to the eye to adjust its growth and/or shape which may correct the eye's central vision, as taught in U.S. Pat. No. 10,429,670, which issued patent is incorporated herein by reference in its entirety.

In one embodiment of the principles described herein, an ophthalmic lens includes a lens body configured to be positioned relative to an eye. The lens body includes an optic zone configured to direct light towards a central region of the retina of the eye. At least one optic feature including a metasurface array of the lens body has a characteristic that selectively directs light into the eye away from the central region of the retina.

The optic feature can be formed on either an anterior or a posterior surface of the ocular lens. In examples where the lens body is made of multiple layers, the optic feature can be formed on an internal or external surface of any one of the layers. Such an internal or external surface can be on an intermediate layer or on another surface of an anterior layer or a posterior layer. In some exemplary embodiments, the metasurface array can be incorporated into the lens body without affecting the ocular lens' field of curvature. The metasurface array can also be one of multiple independent metasurface arrays or locations that are incorporated into the ocular lens and are independently tuned to direct light towards specific areas of the retina. Such optic features can have different sizes, be tuned to different wavelengths of light, can include different cross-sectional shapes, different refractive indexes, different focusing powers, other differing characteristics, or combinations thereof.

FIG. 4C is cross sectional view of one embodiment of an ocular lens 10 directing light into an eye 12 according to the principles of the present disclosure. In this example, the ocular lens 10 is placed over the eye 12. Ambient light rays 14, 16, 18 enter the eye 12 after having passed through the ocular lens 10. These rays of light are focused by an optic zone 20 of the ocular lens 10 towards a central region 22 of the retina 24. The focal point 25 of the light rays 14, 16, 18 is formed on the central region 22 of the retina 24, which causes the eye to clearly see objects that are both near and far from the eye.

Other ambient light rays 26, 28, 30 also enter the eye 12 through the ocular lens 10. These light rays 26, 28, 30 are refracted differently than light rays 14, 16, 18. Light rays 26, 28, 30 are directed towards the peripheral region 32 of the retina 24. In the example of FIG. 4C, the light rays 26, 28, 30 are focused on the peripheral region 32 of the retina 24. This may cause the eye 12 to have a stimulus that indicates that the focusing power of the eye and the axial length 34 are balanced. Thus, the eye 12 may be induced to maintain its current ratio between the focusing power and axial length 34.

Light rays 26, 28.30 are refracted differently than light rays 14, 16, 18 because light rays 26, 28, 30 pass through the ocular lens 10 at a metasurface array 36 that has a different refractive property than the refractive properties in the optic zone 20 of the ocular lens 10. The metasurface arrays 36 are illustrated as protrusions for ease of explanation and identification in the figures only. As noted above, the metasurface arrays do not substantially or noticeably alter the surface profile geometry of the ophthalmic lens or the thickness of the lens. According to one exemplary embodiment, the metasurface array 36 can be create a positive or negative refraction, depending on the geometry of the array, such as the angle of incidence, wavelength, and period of the array. The metasurface array 36 can be an active or a passive metasurface array. The metasurface array 36 may be formed according to the processes disclosed herein

In some examples, the ocular lens 10 is a contact lens, a soft contact lens, a rigid gas permeable contact lens, an implantable contact lens, another type of lens, or combinations thereof. Alternatively, the ocular lens 10 can be any ophthalmic lens including a lens for spectacles. In the example of FIG. 4C, the optic zone 20 is free of the metasurface array 36 or includes a metasurface array configured to direct light on the central region 22 of the retina 24. As a result, there is little to no effect from the feature to the eye's central vision. However, multiple, independent metasurface arrays 36 divert some of the light contacting the ocular lens 10 in non-optic regions that would not otherwise enter the eye, or would enter the eye in a different manner Thus, an increased amount of light enters the eye 12 due to the off-axis positioning of the metasurface arrays 36. At least most of the light rays that would otherwise enter the eye and travel towards the peripheral region 32 of the eye 12 without the metasurface arrays 36 continue to enter the eye 12 without aid of the metasurface arrays 36. This light already provides visual feedback to the eye that affects eye growth. However, the additional light redirected by the metasurface arrays 36 into the eye can be controlled to counteract that visual feedback, to enhance that visual feedback, to modify that visual feedback, or otherwise provide a stimulus that affects to eye growth. The additional visual feedback can be used to control myopia progression or, in some cases, prevent myopia from occurring. The amount of light directed towards the peripheral region 32 of the retina 24 may be selected based on the amount of light needed to obtain the desired effect on the eye growth. In some cases, minor amounts of additional light directed from the metasurface arrays 36 are sufficient to achieve the desired results. However, in other cases, directing more light may be beneficial to overcome a strong natural stimulus that causes undesirable axial length growth.

FIG. 4D is cross sectional view of one embodiment of an ocular lens 10 directing light into an eye 12 according to the principles of the present disclosure. In this example, the metasurface arrays 36 direct the light towards the peripheral region 32 of the retina, but the focal point 25 of the directed light is formed in front of the retina 24. Thus, the light rays 26, 28, 30 directed by the metasurface arrays 36 cause a peripheral myopic condition. Such a stimulus may indicate stopping or slowing the growth of the axial growth of the eye 12. In some examples, such a peripheral myopic stimulus may provide a stronger stimulus to the eye 12 to change the eye's growth, without adversely affecting the user's vision since the light in the optic zone is correctly focused on the retina. In some example, directing the redirected light rays 26, 28, 30 to focus short of the peripheral region 32 of the retina 24 may be desirable to treat cases of myopia because such a stimulus indicates that the axial length 34 is too long.

FIG. 4E is cross sectional view of one embodiment of an ocular lens 10 directing light into an eye 12 according to the principles of the present disclosure. In this example, the metasurface arrays 36 direct the light towards the peripheral region 32 of the retina, but the focal point 25 of the directed light is formed behind the retina 24. Thus, the light rays 26, 28, 30 directed by the metasurface arrays 36 cause a peripheral hyperopic condition. Such a stimulus may indicate to increase the axial growth of the eye 12. In some examples, such a peripheral hyperopic stimulus may provide a stimulus to the eye 12 to change the eye's growth rate. In some examples, directing the redirected light rays 26, 28, 30 to focus behind of the peripheral region 32 of the retina 24 may be desirable to treat cases of hyperopia because such a stimulus may signal that the axial length 34 is too short. Similar to the embodiment illustrated in FIG. 4D, the desired stimulus of FIG. 4E is provided outside the optic zone and the user's immediate optical experience is not adversely affected.

While FIGS. 4C-4D have been described with reference to focusing the redirected light within a three dimensional space with reference to the retina 24, the metasurface arrays 36 may direct light into the peripheral space of the vitreous chamber 40 of the eye 12 for any appropriate reason. For example, the light may be directed into the peripheral space without a predetermined focus. In other examples, the light may be directed into the peripheral space with a predetermined focus as described in FIGS. 4C-4E. In some cases, the light may be directed into the peripheral space of the vitreous chamber 40 for treating conditions other than myopia and hyperopia. For example, the light may be directed into the peripheral space for treating other conditions, for entertainment purposes, for communicating with a device implanted in the eye, for other purposes, or combinations thereof.

Further, FIGS. 4C-4E are depicted with a limited number of metasurface arrays directing light to limited areas of the retina for illustrated purposes. Multiple, independent metasurface arrays can focus light to multiple areas of the retina. Each of the independent metasurface array can be customized to specific circumstances of the eye. For example, some of the metasurface arrays may include varying degrees of focusing power, refractive properties, shapes, sizes, materials, thicknesses, other physical characteristics, geometric parameters, angles of incidence, wavelengths, periods, or combinations thereof. Different metasurface arrays of the same ocular lens may independently focus light in front of, on, or behind the retina. In other examples, different areas of the retina receive different intensities of redirected light.

In some examples, the metasurface arrays are constructed so that the wavelengths of the redirected light are not separated. In other words, the features may direct the all wavelengths within the visual light spectrum together. However, in some examples, at least some of the metasurface arrays may be constructed to redirect just selected wavelengths of light towards to the peripheral areas of the retina. As illustrated in the exemplary illustrations of FIGS. 4C-4F, the metasurface arrays are illustrated as protrusions for ease of explanation and identification only. As noted above, the metasurface arrays do not substantially or noticeably alter the surface profile geometry of the ophthalmic lens or the thickness of the lens.

FIG. 4F is a perspective view of one embodiment of an ocular lens 10 with metasurface arrays 36 for directing the light off axis towards a peripheral region of the retina according to the principles of the present disclosure. In this example, the ocular lens 10 includes an optic zone 20 and a non-optic region 92. The metasurface arrays 36 are formed in the non-optic region 92.

As illustrated in FIG. 4F, the optic zone 20 is configured to focus central light 96 passing through the optic zone on the retina 24 in the central region 22 of an eye on which the ocular lens 10 is worn. The optic zone 20 is positioned in front of the eye's pupil. Often, the non-optic region 92 circumscribes the optic zone 20 and makes up the remainder of the ocular lens 10. This non-optic region 92 may be positioned over the iris and, in some cases, portions of the conjunctiva and sclera of the eye. Traditionally, light passing through the non-optic region 92 of the ocular lens 10 does not enter the eye because such light rays would make contact with regions of the eye that do not permit light to enter, such as the iris and sclera. However, in contrast to traditional lenses, the metasurface arrays 36 incorporated into the ocular lens 10 direct peripheral light rays 98 (that would not otherwise be on a trajectory to enter the eye) into the pupil at an angle that, by design, directs the peripheral light towards the peripheral region 32 of the retina 24.

The peripheral light 98 redirected into the eye may not affect the central vision of the eye because the peripheral light 98 is directed into the peripheral region 32 of the retina where peripheral vision is processed. Consequently, the peripheral light 98 that is directed towards the peripheral region 32 of the retina 24 can be intentionally defocused to provide a desired stimulus to the eye. For example, the redirected peripheral light 98 may be focused exactly on the retina. In some cases, such a stimulus may indicate that the eye's axial length is properly proportioned with the eye's focusing power. In other examples, the redirected light rays 98 are focused to fall short of the retina. In some cases, such a stimulus indicates that the eye's axial length is too long for the eye's focusing power, thereby slowing or ceasing the axial growth of the eye. In yet other cases, the redirected light rays 98 can be focused behind the retina, which may create a stimulus that indicates the eye's axial length is too short for the eye's focusing power. Depending on the eye's ability to grow, the eye may be caused to grow in such a manner to at least partial improve the balance between the axial length of the eye and the eye's focusing power based on the stimulus.

The amount of light that is redirected to the peripheral region 32 of the retina 24 is based on the number of the metasurface arrays 36, the refractive index of the metasurface arrays 36, the shape or geometries of the metasurface arrays 36, other factors, and combinations thereof. An ocular lens 10 may be customized for conditions of the eye. For example, in cases where a professional feels that a strong stimulus is desirable, more metasurface arrays 36 may be added to the ocular lens to redirect more light or the focusing power of selected features may be increased. In other examples, a material with certain refractive indexes or features with different shapes may be used to achieve the desired strength of the stimulus. Likewise, these parameters may be scaled down to reduce the strength of the stimulus as desired based on a different eye's condition.

The use of metasurface arrays provides a great deal of flexibility and programmability to the design of the ocular lens. Various metasurface arrays can be on some or all of one or more surfaces of the ocular lens, allowing the lens designer to tune some metasurface arrays to maximize a visible optical effect, while allowing other metasurface arrays to be tuned to provide stimuli to the ocular system.

Turning to FIGS. 5A-5C, an ophthalmic lens 500 is shown. The ophthalmic lens 500 can be an intraocular lens or device, such as the ophthalmic lens 400 described above with respect to FIGS. 4A-4B. In this regard, the ophthalmic lens 500 can include a lens body 504, a metasurface array 550, and certain haptic features; redundant explanation of which is omitted here for clarity.

The ophthalmic lens 500 can be configured to maintain an optical characteristic (e.g., a focal point, an aberration characteristic, a glare characteristic, and so on) subsequent to the physical manipulation of the lens 500 for surgical association with an eye. To facilitate the foregoing, the metasurface array 550 can include metasurface building elements 570 having a defined arrangement, as shown in example of FIG. 5A. The metasurface building elements 570 can, in certain circumstances, maintain the defined arranged subsequent to the manipulation during surgery. Additionally or alternatively, the metasurface building elements 570 can be arranged on the lens 504 in a manner suitable for pre-installation (e.g., before physical manipulation for surgery), such that upon association of the lens body 504 with the eye, the metasurface building elements 570 are arranged in a configuration that produces a desired optical effect.

With reference to FIG. 5B, the lens ophthalmic lens 500 is shown in a folded configuration. By way of particular example, the ophthalmic lens 500 is shown substantially rolled, as can facilitate the introduction of the lens 500 through an incision during surgery. It will be appreciated, however, that the rolled configuration shown in FIG. 5B is for purposes of illustration. In other cases, other configuration can be used to introduce the lens 500 through an incision, including different folds, partial folds, more compact rolls, and so on, in order to physically reduce the footprint of the lens 500 during its association with an eye.

To illustrate, FIG. 5B includes a sample eye 590. The eye 590 can be of a user undergoing cataract surgery, for example. The eye 590 can have a geometry or profile 591. The profile 591 can correspond to many different attributes of the eye 590, and the lens 500 can be adapted to match or otherwise fit the profile 591, as may be appropriate for a given configuration. As such, the profile 591 can include information about the eye 590 being rotationally symmetric or rotationally non-symmetric, and the lens 504 can have an appropriate associated geometry, which can be manufactured according to the methods of FIGS. 8A-10B, described herein.

The eye 590 can be undergoing a surgery procedure. As such, FIG. 5B shows an incision 592. Incision 592 is shown for purposes of illustration, including it orientation relative to features of the eye. The location of incision 592 can depend on a variety of factors, such as the procedure type and so on. The incision 592 is shown having a length 593. The length 593 can correspond to the total size or the longest size of the opening into a region of the eye 590 whereat the lens 500 is to be installed. Reducing the value of the length 593 can be desirable, for example, in order to reduce the risk of infection or other complications. Accordingly, the lens 500 can be adapted in order to minimize the value of the length 593. For example, the length 593 can be substantially between about 1 mm to 2 mm. In some cases, the length 593 can be less than 1 mm, such as being less than 0.75 mm. In other cases, the length 593 can be greater than 2 mm, such as being less than 2 mm or greater, based on a given application. In this regard, it will be appreciated the incision 592 is shown for purposes of illustration, and that the length of the incision 592 in the context of the embodiments described herein, can be proportionally smaller or larger than the incision 592 shown in FIG. 5B.

In this regard, the lens 500 can be rolled or folded, as shown in FIG. 5B, for insertion through the incision 592. In one embodiment, the lens 500 can be inserted through the incision 592 via a needle injection or other minimally invasive insertion procedure. More particularly, the lens 500 can be rolled or folded such that the lens 500 has width (in the folded configuration) of less than or substantially equal to the value of the length 593 of the incision 592. As explained herein, this physical manipulation of the lens 500 can be tailored to support the resulting optical modification of the lens via the metasurface array. For example, the physical manipulation of the lens 500 into a configuration in which the lens can advance through the incision 592 may not hinder the operation of the metasurface array upon unfolding and association of the lens 500 with the eye 590.

To illustrate the foregoing, FIG. 5C shows the ophthalmic lens 500 associated with the eye 590. The configuration shown in FIG. 5C can be representative of a post-folding surgical step. For example, the lens 500 can be inserted through the incision 592 of FIG. 5B, and subsequently be unfolded and arranged appropriately on the eye 590. With this arrangement, the metasurface building elements 570 can be positioned in order to modify optical properties of the lens 500, as desired, including adapting optical properties to provide certain vision and/or disease treatment benefits. FIG. 5C shows that the metasurface building elements 570 can be arranged in substantially the same configuration as that of FIG. 5A. For example, the metasurface building elements 570 can be sufficiently durable (in connection with an optical matrix layer, such as matrix 154 of FIG. 1D) so that upon the unfolding of the lens body 504, the metasurface building elements 570 substantially return to the initial arrangement, e.g., such as that shown in FIG. 1A. Additionally or alternatively, the metasurface building elements 570 can be modified as a result of the folding process and/or the process of associating the lens body 504 with the eye 590. In this manner, the metasurface building elements 570 can be arranged in the embodiment of FIG. 5A to account for this modification, thus being adapted to induce the desired optical property in the installed configuration of FIG. 5C.

The ophthalmic lenses and devices of the present disclosure can also be used in the context of a contact lens, such as an external contact lens that is associated with an eye by the user. This can include, for example, rigid gas permeable ocular lens or scleral lens, as possible examples. The contact lens can also be susceptible to physical manipulation, such as that caused by a user associating the lens with the eye, including pinching the lens, rolling or partially rolling, or other physical manipulations. In this regard, the metasurface array associated with context lens-type embodiments can be configured to maintain the modified optical property of the lens after physically manipulating the lens body for use with the eye. For example, the array can include metasurface building elements or other metasurface features that are arranged to account for the manipulation of the lens, and thus induce the appropriate optical effect after the manipulation.

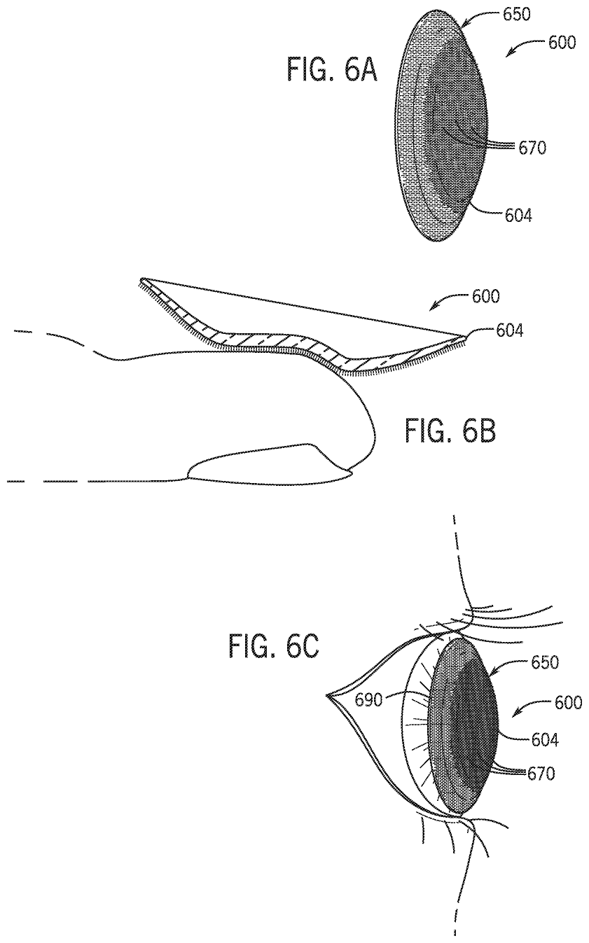

To illustrate the foregoing, FIGS. 6A-6C show an embodiment of an ophthalmic lens 600 according to embodiment of the present disclosure. The ophthalmic lens 600 is manipulateable in order to be used with an eye, such as being used externally on the eye. The ophthalmic lens 600 can be substantially analogous to the ophthalmic lens described herein, and including a lens body 604, a metasurface array 650 and metasurface building elements 670; redundant explanation of which is omitted herein for clarity.

With reference to FIG. 6A, the ophthalmic lens 600 is shown in a pre-installed configuration. The metasurface building elements 670 are shown in FIG. 6A in an arrangement configured to produce a desired optical effect for the ophthalmic lens 600. Additionally or alternatively, the metasurface building elements 670 can be arranged on the lens body 604 in a manner for pre-installation, such that upon association of the lens body 604 with the eye, the metasurface building elements 670 are arranged in a configuration that produces a desired optical effect.

With reference to FIG. 6B, the ophthalmic lens 600 is shown in a configuration in which the lens body 604 is physically manipulated. The degree of physical manipulation is shown enlarged in FIG. 6B for purposes of illustration. In other cases, the deformation and movement of the lens body 604 can be less. The state of physical manipulation of the ophthalmic lens 600 shown in FIG. 6B can correspond to a state in which a user is handling the lens 600 for association with eye, among other possibilities.

With reference to FIG. 6C, the ophthalmic lens 600 is shown in an externally installed configuration with a sample eye 690. In the installed configuration of FIG. 6C, the metasurface array 650 is shown as having the metasurface building elements 670 in substantially the same configuration as that of the metasurface building elements 670 of FIG. 6A. In this manner, the metasurface building elements 670 can operate to modify the optical property of the lens body 604 in a desired manner after the physical manipulation of the lens body 604 shown in FIG. 6B. Additionally or alternatively, the metasurface building elements 670 can be modified as a result of the physical manipulation of FIG. 6B and/or the process of the associating the lens body 604 with the eye 690. In this manner, the metasurface building elements 670 can be arranged in the embodiment of FIG. 6A to account for this modification, thus being adapted to induce the desired optical property in the installed configuration of FIG. 6C.

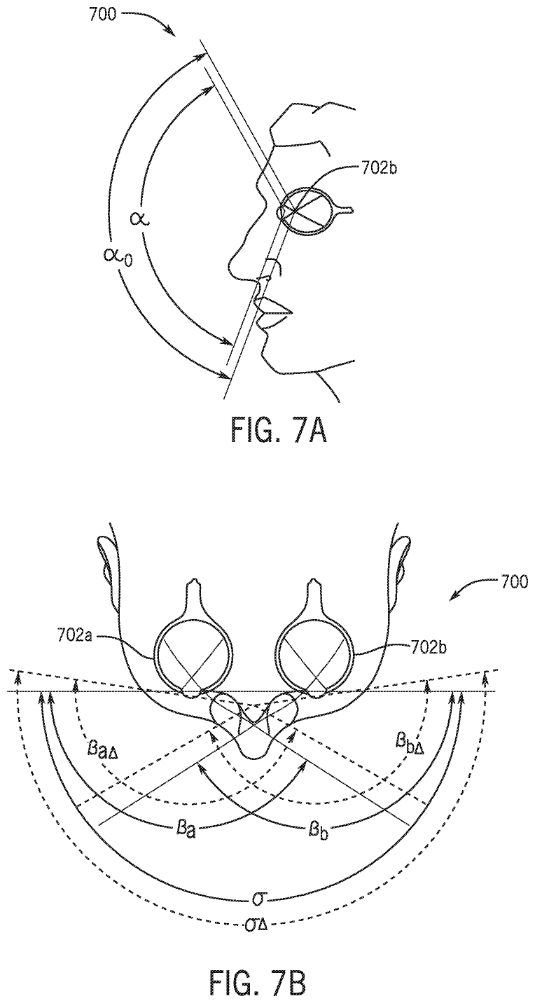

In some embodiments, the metasurface array can be adapted to enhance a field of view of a given patient. For example, the metasurface arrays described herein can be tuned in order to expand or enlarge a field of view as compared with a standard lens. FIGS. 7A and 7B depict a patient 700 having a left eye 702a and a right eye 702b. FIG. 7A shows a vertical field of view α. The vertical field of view α can be approximately 150 degrees, as one example. FIG. 7A further shows an expanded vertical field of view α_{Δ}. The expanded vertical field of view α_{Δ} may be greater than 150 degrees, or otherwise greater than the value of the vertical field of view α, such as being 151 degrees, 153 degrees, 160 degrees, or greater. The expanded field of view α_{Δ} may correspond to a vertical field of view induced by the metasurface arrays described herein. For example, the patient 700 may associate a contact or other lens with the right eye 702b that includes a metasurface array (e.g., the ophthalmic lens 100), thus allowing the patient to experience the expanded vertical field of view α_{Δ}.

With reference to FIG. 7B, the left eye 702a is shown as having a horizontal field of view βₐ and the right eye 702b is shown as having a horizontal field of view β_{b}. The left eye 702a and the right eye 702b are shown as having a combined horizontal field of view σ. The horizontal field of views βₐ, β_{b} can be approximately 150 degrees, as one example. The combined horizontal field of view α can be approximately 180 degrees. The metasurface arrays described herein can also be used to enhance or otherwise modify a horizontal field of view βₐ, and the right eye 702b is shown as having a horizontal field of view β_{b}. In this regard, the metasurface array may allow the patient 700 to have an enhanced or otherwise modified horizontal field of view as compared to wearing a standard contact lens. To illustrate the foregoing, FIG. 7B further shows an expanded horizontal field of view β_{aΔ}, an expanded horizontal field of view β_{bΔ}, and a combined expanded horizontal field of view σ_{A} The expanded horizontal field of views β_{aΔ}, β_{bΔ} can be greater than 150 degrees, or otherwise greater than the value of the horizontal field of view βₐ, β_{b}, such as being 151 degrees, 153 degrees, 160 degrees, or greater. The expanded horizontal field of views β_{aΔ}, β_{bΔ} may correspond to a horizontal field of view induced by the metasurface arrays described herein. For example, the patient 700 may associate a contact or other lens with the right eye 702b that has a metasurface array (e.g., the ophthalmic lens 100), thus allowing the patient to experience the expanded horizontal field of views β_{aΔ}, β_{bΔ}. The expanded horizontal field of views β_{aΔ}, β_{bΔ} may cooperate to define the combined expanded horizontal field of view σ_{Δ}.

The ophthalmic lenses of the present disclosure having metasurface arrays can be manufactured using a variety of appropriate techniques. The ophthalmic lenses can be manufactured in order to produce metasurface arrays having metasurface building elements or other metasurface features that are tuned to induce a specified optical characteristic in the lens. This can include manufacturing techniques that can produce metasurface building elements having a dimension, such as a height dimension that is of, or less than, a cycle wavelength of light. The manufacturing techniques herein can also adapt and associate the metasurface array for a variety of different lens contexts or embodiments. For example, the manufacturing techniques can be used to produce lens for intraocular lens, such as those associatable with an eye during surgery. In another context, the techniques can be used for substantially external lenses, such as contact lenses, including rigid gas permeable ocular lenses, scleral lenses, spectacle lenses, and so on. As such, to the extent that the following methods are discussed as generally being used to manufacture an embodiment of an ophthalmic lens, it will be appreciated that the manufacturing techniques can also be used to produce other ophthalmic lenses, as contemplated herein. FIGS. 8A-8D depict operations for using a molding process for forming one or more of the ophthalmic lens of the present disclosure. Generally, a metasurface array can be formed and subsequently associated with a precursor form of a lens body during a molding process. The precursor form of the lens body can be a liquid lens material, for example, that is subsequently pressed or shaped during a molding process, and cured for finishing. In this regard, the molding process of FIGS. 8A-8D can allow the metasurface array to be associated with a non-solid substrate. The molding process can be desirable, for example, by implementing a standardized mold shape for producing groups of standardized lens bodies. A metasurface array can be associated with the mold that contains a desired arrangement of metasurface building elements or other metasurface features in order to induce the desired optical effects for the standardized lens body.

With reference to FIG. 8A, an illustrative cross-sectional view of an operation of forming one or more the ophthalmic lens of the present disclosure. In FIG. 8A, a molding apparatus 830 is shown. The molding apparatus 830 can include a first mold portion 832a and a second mold portion 832b. The first mold portion 832a and the second mold portion 832b can be operable to move relative to one another, for example, to distribute and shape material disposed therebetween. The first and second mold portions 832a, 832b can also be associated with a variety of systems that introduce material for molding, including certain extrusion-type systems.

In the embodiment of FIG. 8A, the first mold portion 832a can be configured to receive a metasurface array, such as any of metasurface arrays and variations described herein. In this regard, FIG. 8A shows a metasurface array 850. The metasurface array 850 can be manufactured separately from the lens body. For example, the metasurface array 850 can include a variety of metasurface building elements 860 arranged in a matrix 864 or other material that can allow the metasurface array 850 to be associated with a non-solid substrate. As described herein, the matrix 864 can be formed from a polydimethylsiloxane (PDMS) substrate. The matrix 864 can help hold the metasurface building elements 860 in a desired orientation. The matrix 864 can also be used to associate the metasurface building element 860 with a non-solid substrate, as shown in FIG. 8B. While many techniques can be used to form the metasurface array 850, in one embodiment, the metasurface array 850 can be formed using an etching process, in which an amorphous silicon layer is patterned to form the metasurface building elements 860. In other cases, other techniques can be used to form the metasurface building elements 860, including techniques that form the metasurface building elements 860 at least partially from a silver dioxide or titanium dioxide material.

With reference to FIG. 8B, an illustrative cross-sectional view of another operation of forming one or more the ophthalmic lens of the present disclosure is shown. In the embodiment of FIG. 8B, a liquid lens material 844 is introduced to the molding apparatus 830. The liquid lens material 844 can be introduced to the molding apparatus 830 via an extrusion process, in certain embodiments. The liquid lens material 844 can be a precursor form of a lens body for one or more of the ophthalmic lens of the present disclosure. In this regard, the liquid lens material 844 can be made from any material suitable for use in lens bodies. For example, the liquid lens material 844 can be made of any material that is rigid and gas or oxygen permeable when cured, polymerized, or hardened. In some embodiments, the liquid lens material 844 can include a monomer or polymer material. In some embodiments, the liquid lens material 844 can include siloxane material. In some embodiments, liquid lens material 844 may include an acrylate material. In some embodiments, liquid lens material 844 may include cellulose acetate butyrate, siloxane acrylates, t-butyl styrene, flurosiloxane acrylates, perfluroethers, other types of polymers, or combinations thereof. These materials may include various combinations of monomers, polymers, and other materials to form the final polymer. For example, common components of these materials may include HEMA, HEMA-GMA, and the like.

FIG. 8B shows the liquid lens material 844 contacting the metasurface array 850. In this manner, the metasurface array 850 can be associated with a non-liquid substrate. The volume of material supplied for the liquid lens material 844 can be based on the size and physical characteristics of the target lens body, and thus can be standardized.

With reference to FIG. 8C, the molding apparatus 830 is shown in a configuration in which the first mold portion 832a and the second mold portion 832b are moved relative to one another. In particular, the second mold portion 832b can be configured to press the liquid lens material 844 against the metasurface array 850. The second mold portion 832b can thus distribute the liquid mold material 844 across the metasurface array 850 and cause the liquid lens material 844 to substantially assume a shape defined by the first and second mold portions 832a, 832b, as shown in FIG. 8C. Upon assuming the desired shape, the liquid lens material 844 can be cured or otherwise hardened to produce the ophthalmic lens of the present disclosure.

In this regard, FIG. 8D shows an ophthalmic lens 800 produced using the molding apparatus show in FIGS. 8A-8C. The ophthalmic lens 800 can optionally undergo one or more post-molding finishing processes, for example, in order to produce the lens 800 shown in FIG. 8D having a lens body 804 formed from the liquid lens material 844. For example, one or more surfaces can be polished and/or further shaped using various precision tooling. The ophthalmic lens 800 can also undergo a chemical bath or other form of treatment to finish one or more surface of the lens 800. This can include causing at least of the matrix 864 to be removed from the ophthalmic lens 800, such as can be the case where the matrix is sacrificial matrix, however, this is not required.

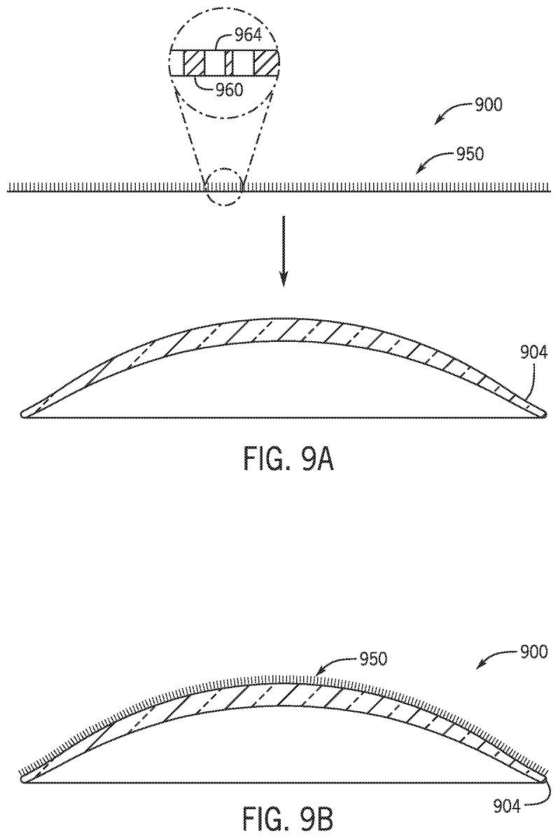

FIGS. 9A-9B depict another embodiment of manufacturing techniques for forming one or more of the ophthalmic lens of present disclosure. In particular, FIGS. 9A-9B show forming a metasurface array, such as the various metasurface arrays and embodiment thereof described herein, as a peelable sheet. The peelable sheet can be configured to adhere the metasurface array to an outer surface of a lens body of the various ophthalmic device described herein. In this regard, the metasurface array and the lens body can be formed or otherwise manufactured separately, such as via separate processes, and subsequently associated. This can enhance the adaptability of the ophthalmic lens manufacturing techniques, for example, by manufacturing a batch or group of standardized lens bodies, and subsequently associating each of the group of standardized lens bodies with a metasurface array, as needed, and as tuned for a target optical effect of the lens body.

With reference to FIG. 9A, an illustrative cross-sectional view of an operation of associating a metasurface array with a lens body is shown. In particular, a metasurface array 950 is shown being advanced toward a lens body 904. The lens body 904 and the metasurface array 950 can be substantially analogous to the various bodies and arrays described herein; redundant explanation of which is omitted here for clarity.

The metasurface array 950 can be configured to adhere to the lens body 904. For example, the metasurface array 950 can define one or more peelable sheets that is associatable with the lens body 904 is a manner that substantially mitigates subsequent separation. To facilitate the foregoing, the array 950 can include metasurface building element 960 arranged in a matrix 964. The matrix 964 can be adapted to associate the metasurface building elements 960 with the outer surface of the lens body 904. For example, the matrix 964 can have certain adhesive properties that cause the metasurface array 950 to maintain contact with the outer surface of the lens body 904. Additionally or alternatively, the matrix 964 can be user to define a surface to receive an adhesive treatment, laminate, or other layer, coating, and so on to facilitate the association of the array 950 with the lens body 904.

The metasurface array 950 and the lens body 904 can be associated with one another in order to form an ophthalmic lens 900. The ophthalmic lens 900 can be one or more of the ophthalmic lenses described herein, which use metasurface features to modify an optical characteristic of a lens. In this regard, FIG. 9B shows the ophthalmic lens 900 subsequent to association of the array 950 and body 904 shown in FIG. 9A. As discussed in relation to FIG. 8D, the ophthalmic lens 900 can be subjected to one or more treatment processes subsequent to associating the array and the lens body. FIG. 9B shows at least some of the matrix 964 removed. In other embodiments, the lens 900 can be subjected to other treatment procedures, including polishing and various chemical treatments.

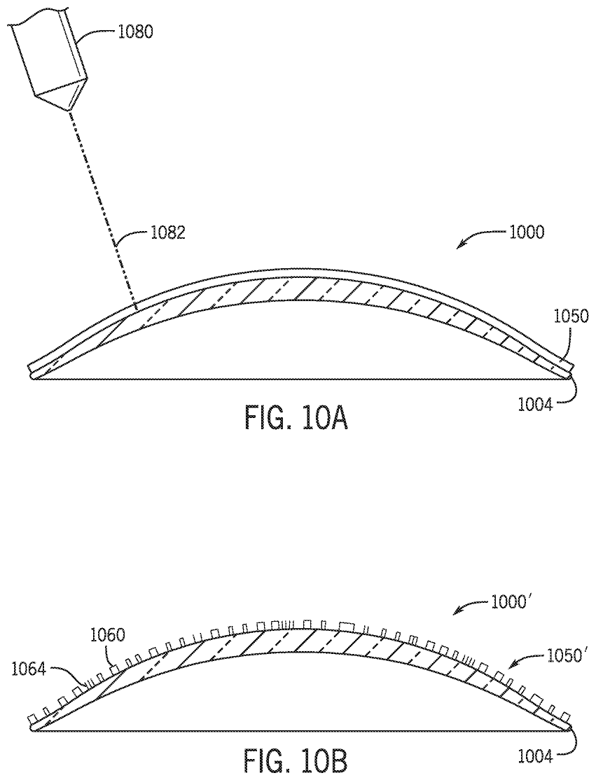

FIGS. 10A-10B depict another embodiment of manufacturing techniques for forming one or more of the ophthalmic lens of present disclosure. In particular, FIGS. 10A-10B show forming a metasurface array on or otherwise directly in contact with a lens body. For example, the lens body can be manufactured by one or processes, and metasurface features can be patterned directly on the lens body. In this regard, the metasurface features are manufactured subsequent to the production of the lens body.

With reference to FIG. 10A, an illustrative cross-sectional view of an operation of establishing a metasurface array on a lens body is shown. In particular, FIG. 10A shows an ophthalmic lens 1000 during an operation of manufacture. The ophthalmic lens 1000 can be one or more of the ophthalmic lens described herein, including being one or more intraocular lenses or substantially external contact lenses.

The ophthalmic lens 1000 can include at least a lens body 1004 and a metasurface array 1050. The metasurface array 1050 can initially be formed from one or more base materials, such as an amorphous silicon layer that is overlaid onto the lens body 1004. The metasurface array 1050 can receive electromagnetic radiation or other input in order to pattern the amorphous silicon layer such that the array 1050 includes metasurface building elements in a desired configuration. In this regard, FIG. 10A shows an instrument 1080 that is configured to emit energy along a path 1082. In some cases, the instrument 1080 can be configured for etching and/or lithography. Upon etching, one or more matrix layers, binders, substrates or the like can be subsequently added to the metasurface array 1050 in order to facilitating maintain the metasurface building elements in the patented orientation.

With reference to FIG. 10B, the ophthalmic lens 1000 subsequent to the patterning operation shown in FIG. 10A. In particular, FIG. 10B shows metasurface building elements 1060 in an arrangement resulting from the patterning performed by the instrument 1080. The ophthalmic lens 1000 also include a matrix material 1064, which can be used to maintain the orientation of the metasurface building elements during use, such as maintain the orientation after folding the lens or other physical manipulations.

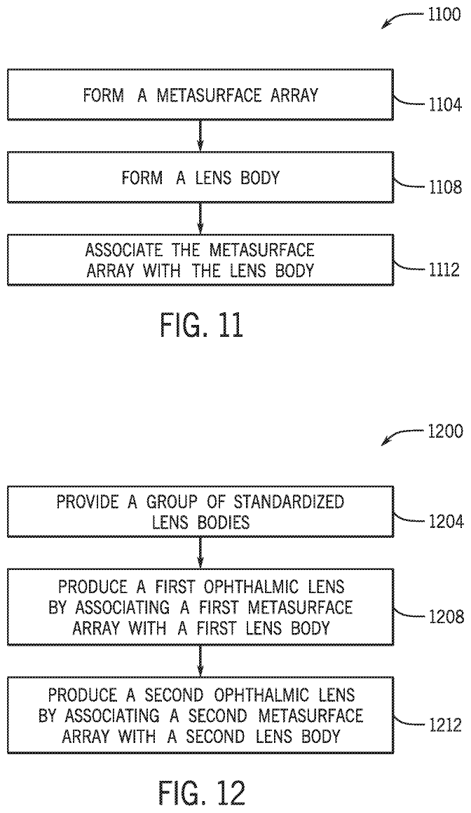

To facilitate the reader's understanding of the various functionalities of the embodiments discussed herein, reference is now made to the flow diagrams in FIGS. 11 and 12, which illustrates process 1100 and 1200, respectively. While specific steps (and orders of steps) of the methods presented herein have been illustrated and will be discussed, other methods (including more, fewer, or different steps than those illustrated) consistent with the teachings presented herein are also envisioned and encompassed with the present disclosure.

With reference to FIG. 11, process 1100 generally relates to manufacturing an ophthalmic lens. The process 1100 can be used to produce the various ophthalmic lens and devices described herein, for example, such as the ophthalmic lenses 100, 200, 300, 400, 500, 600, 700, 800, 900, and 1000 and variations and embodiments thereof.

At operation 1104, a metasurface array can be formed by establishing metasurface building elements in a matrix. For example and with reference to FIG. 8A, metasurface building elements 860 can be established in a matrix 864. The metasurface building elements 860 can be patterned, such as via an etching process, in order to define various metasurface features have characteristics tuned modify optical characteristics of an associated lens. FIG. 10A shows an example instrument 1080 that used to pattern an amorphous silicon layer to form the metasurface building elements as nano-posts having dimensions that are smaller than a cycle wavelength of light. In other embodiments, other techniques can be used to form the metasurface building elements in a matrix.

At operation 1108, a lens body can be formed having a profile shaped to match a geometry of an eye. For example and with reference to FIG. 8B, a liquid lens material 844 can be introduced into the molding apparatus 830. The first and second mold portions 832a, 832b can cooperate to form the liquid lens material 844 into a lens shape, such as that which has a profiled shaped to match a geometry of an eye. For example, FIG. 8C shows the liquid lens material 844 being distributed along the metasurface array 850 and into a lens shape, when the first and second mold portions 832a, 832b move toward one another.

At operation 1112, the metasurface array can be associated with the lens body, thereby forming an ophthalmic lens, such as a foldable ophthalmic lens as described herein. For example and with reference to FIGS. 8A-8C, the metasurface array 850 can be associated with a non-solid substrate, such as the liquid lens material 844. The liquid lens material 844 can be or form a portion of a precursor form of the lens body 804. As such, the liquid lens material 844 can be subsequently cured or otherwise hardened to form the lens body 804 and thus facilitate the association of the lens body 804 and the metasurface array 850. As another example, and with reference to FIGS. 9A-9B, the metasurface array 950 can be a peelable sheet that is associated with the lens body 904. For example, the metasurface array 950 can have or be associated with certain adhesive properties and thus be associate with the lens body 904 in a manner that mitigates separation of the array 950 and the body 904 during use of the lens 900. Another example, and with reference to FIGS. 10A-10B, the metasurface array 1050 can be formed directly on the lens body 1004. The resulting lens can be foldable, such as being foldable or rollable for introduction through an incision and into a region of the eye during surgery. In certain cases, the metasurface array is adapted to establish at least one of a low aberration characteristic, a low glare characteristic, or an enhanced contrast characteristic of the foldable ophthalmic lens in an installed configuration with the eye.

With reference to FIG. 12, process 1200 generally relates to manufacturing standardized ophthalmic lenses. The process 1200 can be used to produce the various ophthalmic lens and devices described herein, for example, such as the ophthalmic lenses 100, 200, 300, 400, 500, 600, 700, 800, 900, and 1000 and variations and embodiments thereof.

At operation 1204, a group of standardized lens bodies can be provided. For example, and with reference to FIGS. 8A-8C, a group of standardized lens bodies can be produced using a molding apparatus. The first and second mold portions 832a, 832b can be used to form the lens body 804 from a liquid lens material 844. The lens body 804 can thus be one of a group standardized lens bodies. In other cases, the lens bodies can be manufactured using other techniques.

At operation 1208, a first ophthalmic lens can be produced by associating a first metasurface array with a first lens body of the group of standardized lens bodies. For example, and with reference to FIGS. 8A-8C, the lens body 804 and the metasurface array 850 can be components of a first ophthalmic lens. The array 850 can include metasurface building elements having a first configuration that are used to induce a first optical effect for the lens body.

At operation 1212, a second ophthalmic lens can be produced by associating a second metasurface array with a second lens body of the group of standardized bodies. For example, and with reference to FIGS. 8A-8C, the lens body 804 and the metasurface array 850 can be components of a second ophthalmic lens. The array 850 in this operation can thus include metasurface building elements having a second configuration that are used to induce a second optical effect for the lens body.

Other examples and implementations are within the scope and spirit of the disclosure and appended claims. For example, features implementing functions may also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and B and C). Further, the term “exemplary” does not mean that the described example is preferred or better than other examples.

The foregoing description, for purposes of explanation, uses specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An ophthalmic lens, comprising:
a lens body; and
a metasurface array on the lens body and comprising an arrangement of metasurface building elements dimensioned from an optical wavelength and configured across the lens body to define a reduced glare/halo characteristic of the ophthalmic lens.

2. The ophthalmic lens of claim 1, wherein the reduced glare characteristic is maintained after physically manipulating the ophthalmic lens for use with an eye.

3. The ophthalmic lens of claim 1, wherein the arrangement of metasurface building elements are configured across the lens body for contrast enhancement of the ophthalmic lens.

4. The ophthalmic lens of claim 1, wherein the arrangement of metasurface building elements are configured across the lens body to reduce an aberration characteristic of the lens body.

5. The ophthalmic lens of claim 4, wherein the aberration characteristic comprises one or both of a chromatic aberration or a monochromatic aberration.

6. The ophthalmic lens of claim 1, wherein the lens body comprises a field of view, the arrangement of metasurface building elements being configured to modify the field of view.

7. The ophthalmic lens of claim 1, wherein metasurface building elements of the arrangement include dimensions less than a wavelength of light traversing the lens body.

8. The ophthalmic lens of claim 7, wherein the dimensions of the metasurface building elements include a height dimension of the metasurface building elements.

9. The ophthalmic lens of claim 1, wherein arrangement of the metasurface building elements include a collection of nano-posts.

10. The ophthalmic lens of claim 9, wherein the collection of nano-posts include nano-posts of dissimilar shapes.

11. The ophthalmic lens of claim 9, wherein the collection of nano-posts include nano-posts of dissimilar orientations.

12. The ophthalmic lens of claim 9, wherein the collection of nano-posts define a first density of metasurface building elements on a first portion of the lens body and a second density of metasurface building elements on a second portion of the lens body that is different than the first density.

13. The ophthalmic lens of claim 1, wherein the lens body comprises a focal point, the arrangement of metasurface building elements being configured to modify the focal point.

14. The ophthalmic lens of claim 13, wherein the modified focal point is configured to control myopia progression.

15. The ophthalmic lens of claim 1, wherein the ophthalmic lens is an intraocular device.

16. The ophthalmic lens of claim 15, wherein the lens body is substantially flat.

17. The ophthalmic lens of claim 15, wherein the lens includes a portion having a thickness of about 0.25 mm.

18. The ophthalmic lens of claim 1, wherein the ophthalmic lens is a contact lens.

19. The ophthalmic lens of claim 18, wherein the contact lens includes one of a rigid gas permeable ocular lens or a scleral lens.

20. The ophthalmic lens of claim 18, wherein the contact lens is a hybrid lens, including a soft periphery.

21. The ophthalmic lens of claim 18, wherein the contact lens includes a hydrogel component.

22. The ophthalmic lens of claim 18, wherein the contact lens is a molded lens.

23. The ophthalmic lens of claim 1, wherein the ophthalmic lens is at least partially formed from a titanium dioxide material.

24. A method of manufacturing a foldable ophthalmic lens, comprising:
forming a metasurface array by establishing metasurface building elements in a matrix;
forming a lens body having a profile shaped to match a geometry of an eye; and
associating the metasurface array with the lens body to form the foldable ophthalmic lens, the foldable ophthalmic lens being foldable or rollable for introduction through an incision and into a region of the eye during surgery, the metasurface building elements being adapted to establish at least one of a low aberration characteristic, a low glare/halo characteristic, or an enhanced contrast characteristic of the foldable ophthalmic lens in an installed configuration with the eye.

25. The method of claim 24, wherein the incision is less than 2.0 mm.

26. The method of claim 25, wherein the incisions is less than 1.5 mm.

27. The method of claim 24, wherein the lens body is substantially flat prior to being folded or rolled for the introduction through the incision.

28. The method of claim 27, wherein the foldable ophthalmic lens is an intraocular device having a diameter of less than about 6 mm and a thickness of less than about 0.25 mm.

29. The method of claim 24, wherein the operation of associating comprises coupling the metasurface array with a non-solid substrate.

30. The method of claim 29, wherein the non-solid substrate comprises a precursor form of the lens body.

31. The method of claim 24, wherein the operation of associating the metasurface array and the lens body is performed using a molding apparatus.

32. The method of claim 31, wherein the molding apparatus comprises:
a first mold portion configured to receive the metasurface array; and
a second mold portion configured to press a lens material against the metasurface array.

33. The method of claim 32, wherein the lens material comprises a liquid lens material defining a precursor form of the lens body.

34. The method of claim 33, wherein the operation of forming the lens body further comprises distributing the liquid lens material along the metasurface array by combining the first and second mold portions.

35. The method of claim 34, wherein the operation of forming the lens body further comprises curing the liquid lens material to form the lens body.

36. The method of claim 24, wherein the matrix comprises a sacrificial matrix configured to be at least partially removed subsequent to the operation of associating.

37. The method of claim 24, wherein the operation of forming the metasurface array comprises patterning an amorphous silicon layer to form nano-posts defining the metasurface building elements.

38. The method of claim 37, wherein the nano-posts have a dimension of, or less than, an optical wavelength.

39. The method of claim 37, wherein the operation of patterning comprises defining an arrangement of nano-posts tuned to the profile of the lens body.

40. The method of claim 39, wherein the operation of forming the metasurface array further comprises one or both of lithography or dry etching.

41. The method of claim 39, wherein the operation of forming the metasurface array further comprises associating the nano-posts with a matrix material forming the matrix.

42. The method of claim 41, wherein the matrix material comprises polydimethylsiloxane.

43. The method of claim 24, wherein the operation of forming the metasurface array comprises forming a peelable sheet configured to adhere to an outer surface of the lens body during the operation of associating the metasurface array with the lens body.

44. A method of manufacturing standardized ophthalmic lenses, comprising:
providing a group of standardized lens bodies;
producing a first ophthalmic lens by associating a first metasurface array with a first lens body of the group of standardized lens bodies; and
producing a second ophthalmic lens by associating a second metasurface array with a second lens body of the group of standardized lens bodies,
wherein the first and second metasurface arrays have different arrangements of metasurface building elements, thereby inducing differential optical properties for the standardized bodies of the first and second ophthalmic lenses.

45. The method of claim 44, wherein the standardized lens bodies have a portion with a thickness of about 0.25 mm or less.

46. The method of claim 44, wherein the standardized lens bodies are substantially flat.

47. The method of claim 44, wherein the standardized lens bodies include a haptic feature for an intraocular lens.

48. The method of claim 44, wherein the first and second ophthalmic devices are contact lenses, comprising a rigid gas permeable ocular lens or a scleral lens.

49. The method of claim 44, wherein the first ophthalmic lens has a first focal point and the second ophthalmic lens has a second focal point that is different from the first focal point.

50. The method of claim 44, wherein the metasurface building elements of both the first and second ophthalmic lenses have a dimension that is of, or less than, an optical wavelength.

51. The method of claim 44, wherein the operation of producing the first or the second ophthalmic lenses further comprises associating a respective one of the first or second metasurface arrays with a non-solid substrate comprising a precursor form of any of the standardized lens bodies.

52. The method of claim 51, wherein the operation of associating the first or the second metasurface arrays with a respective one of the first or the second lens bodies further comprises distributing the non-solid substrate using a molding process.

53. The method of claim 52, wherein the non-solid substrate is curable to form the respective one of the first or the second ophthalmic lenses.

54. The method of claim 44, wherein the differential optical properties comprises a reduced glare characteristic of the ophthalmic lenses.
